(19) United States
(12) Patent Application Publication
(10) Pub. No.: US 2025/0011783 A1
(43) Pub. Date: Jan. 9, 2025

(54) GENE THERAPY FOR NEURODEGENERATIVE DISORDERS

(71) Applicant: Prevail Therapeutics, Inc., New York, NY (US)

(72) Inventors: Asa Abeliovich, New York, NY (US); Laura Heckman, Philadelphia, PA (US); Herve Rhinn, San Francisco, CA (US)

(73) Assignee: Prevail Therapeutics, Inc., New York, NY (US)

(21) Appl. No.: 18/743,624

(22) Filed: Jun. 14, 2024

Related U.S. Application Data
(62) Division of application No. 16/838,993, filed on Apr. 2, 2020, now Pat. No. 12,049,626.
Application No. 16/838,993 is a continuation of application No. PCT/US2018/054223, filed on Oct. 3, 2018.
(60) Provisional application No. 62/567,303, filed on Oct. 3, 2017.
(60) Provisional application No. 62/567,305, filed on Oct. 3, 2017.

(51) Int. Cl. C12N 15/113; A61K 9/00; A61K 48/00; A61P 25/02; A61P 25/16; A61P 25/28; C07K 14/005; C12N 7/00; C12N 9/24; C12N 15/86

(52) U.S. Cl. CPC C12N 15/113; A61K 9/0019; A61K 9/0085; A61K 48/0075; A61P 25/02; A61P 25/16; A61P 25/28; C07K 14/005; C12N 7/00; C12N 9/2402; C12N 15/86; C12Y 302/01045; A61K 48/00; C12N 2310/141; C12N 2310/531; C12N 2710/14043; C12N 2750/14133; C12N 2750/14143; C12N 2770/32022

(57) ABSTRACT

The disclosure relates, in some aspects, to compositions and methods for treatment of diseases associated with aberrant lysosomal function, for example Parkinson's disease and Gaucher disease. In some embodiments, the disclosure provides expression constructs comprising a transgene encoding one or more inhibitory nucleic acids targeting SCNA or a portion thereof, TMEM106B or a portion thereof, or any combination of the foregoing. In some embodiments, the disclosure provides methods of Parkinson's disease by administering such expression constructs to a subject in need thereof.

### RELATED APPLICATIONS

This application claims the benefit under 35 U.S.C. 119 (e) of U.S. Provisional Application Ser. Nos. 62/567,303, filed Oct. 3, 2017, entitled “GENE THERAPIES FOR LYSOSOMAL DISORDERS”, and 62/567,305, filed Oct. 3, 2017, entitled “GENE THERAPIES FOR LYSOSOMAL DISORDERS”, the entire contents of each of which are incorporated herein by reference.

### BACKGROUND

Gaucher disease is a rare inborn error of glycosphingolipid metabolism due to deficiency of lysosomal acid β-glucocerebrosidase (Gcase, “GBA”). Patients suffer from non-CNS symptoms and findings including hepatosplenomegly, bone marrow insufficiency leading to pancytopenia, lung disorders and fibrosis, and bone defects. In addition, a significant number of patients suffer from neurological manifestations, including defective saccadic eye movements and gaze, seizures, cognitive deficits, developmental delay, and movement disorders including Parkinson's disease.

Several therapeutics exist that address the peripheral disease and the principal clinical manifestations in hematopoietic bone marrow and viscera, including enzyme replacement therapies as described below, chaperone-like small molecule drugs that bind to defective Gcase and improve stability, and substrate reduction therapy that block the production of substrate that accumulate in Gaucher disease leading to symptoms and findings. However, other aspects of Gaucher disease (particularly those affecting the skeleton and brain) appear refractory to treatment.

### SUMMARY

In addition to Gaucher disease patients (who possess mutations in both chromosomal alleles of GBA1 gene), patients with mutations in only one allele of GBA1 are at highly increased risk of Parkinson's disease (PD). The severity of PD symptoms—which include gait difficulty, a tremor at rest, rigidity, and often depression, sleep difficulties, and cognitive decline—correlate with the degree of enzyme activity reduction. Thus, Gaucher disease patients have the most severe course, whereas patient with a single mild mutation in GBA1 typically have a more benign course. Mutation carriers are also at high risk of other PD-related disorders, including Lewy Body Dementia, characterized by executive dysfunction, psychosis, and a PD-like movement disorder, and multi-system atrophy, with characteristic motor and cognitive impairments. No therapies exist that alter the inexorable course of these disorders.

In some aspects, the disclosure is based on expression constructs encoding one or more inhibitory RNA (e.g., shRNA, miRNA, etc.) that targets a PD-associated gene (e.g., α-Synuclein (α-Syn), transmembrane protein 106B (TMEM106B), ribosomal protein s25 (RPS25), microtubule-associated protein tau (MAPT), or a combination thereof). In some aspects, the disclosure is based on expression constructs (e.g., vectors) encoding Gcase (or a portion thereof) and one or more additional gene products from PD-associated genes (e.g., α-Syn). Without wishing to be bound by any particular theory, combinations of gene products described herein act together (e.g., synergistically) to reduce one or more signs and symptoms of PD when expressed in a subject.

Accordingly, in some aspects, the disclosure provides an isolated nucleic acid encoding an inhibitory RNA that targets SNCA (e.g., a portion of SCNA) and inhibits expression and/or activity of α-Syn. In some aspects, the disclosure provides an isolated nucleic acid comprising an expression construct encoding a first gene product and a second gene product, wherein each gene product independently is selected from the gene products, or portions thereof, set forth in Table 1.

Accordingly, in some aspects, the disclosure provides an isolated nucleic acid encoding an inhibitory RNA that targets TMEM106B (e.g., a portion of TMEM106B) and inhibits expression and/or activity of TMEM106B. In some aspects, the disclosure provides an isolated nucleic acid comprising an expression construct encoding a first gene product and a second gene product, wherein each gene product independently is selected from the gene products, or portions thereof, set forth in Table 1.

Accordingly, in some aspects, the disclosure provides an isolated nucleic acid encoding an inhibitory RNA that targets a gene encoding RPS25 (e.g., a portion of a gene encoding RPS25) and inhibits expression and/or activity of RPS25. In some aspects, the disclosure provides an isolated nucleic acid comprising an expression construct encoding a first gene product and a second gene product, wherein each gene product independently is selected from the gene products, or portions thereof, set forth in Table 1.

Accordingly, in some aspects, the disclosure provides an isolated nucleic acid encoding an inhibitory RNA that targets MAPT (e.g., a portion of a gene encoding MAPT) and inhibits expression and/or activity of MAPT. In some aspects, the disclosure provides an isolated nucleic acid comprising an expression construct encoding a first gene product and a second gene product, wherein each gene product independently is selected from the gene products, or portions thereof, set forth in Table 1.

In some embodiments, a first gene product or a second gene product is a Gcase protein, or a portion thereof. In some embodiments, a first gene product or a second gene product is an interfering nucleic acid (e.g., shRNA, miRNA, dsRNA, etc.). In some embodiments, an interfering nucleic acid inhibits expression of α-Synuclein (α-Synuclein). In some embodiments, the first gene product is a Gcase protein, and the second gene product is an interfering nucleic acid (e.g., shRNA, miRNA, dsRNA, etc.) that inhibits expression of α-Syn (e.g., an interfering nucleic acid that targets SCNA).

In some embodiments, an interfering nucleic acid inhibits expression of TMEM106B. In some embodiments, the first gene product is a Gcase protein, and the second gene product is an interfering nucleic acid (e.g., shRNA, miRNA, dsRNA, etc.) that inhibits expression of TMEM106B (e.g., an interfering nucleic acid that targets TMEM106B).

In some embodiments, an interfering nucleic acid inhibits expression of RPS25. In some embodiments, the first gene product is a Gcase protein, and the second gene product is an interfering nucleic acid (e.g., shRNA, miRNA, dsRNA, etc.) that inhibits expression of a gene encoding RPS25 (e.g., an interfering nucleic acid that targets RPS25 encoding sequence).

In some embodiments, an interfering nucleic acid inhibits expression of MAPT. In some embodiments, the first gene product is a Gcase protein, and the second gene product is an interfering nucleic acid (e.g., shRNA, miRNA, dsRNA, etc.) that inhibits expression of MAPT (e.g., an interfering nucleic acid that targets MAPT).

In some embodiments, an expression construct further comprises one or more promoters. In some embodiments, a promoter is a chicken-beta actin (CBA) promoter, a CAG promoter, a CD68 promoter, or a JeT promoter. In some embodiments, a promoter is a RNA pol II promoter or a RNA pol III promoter (e.g., U6).

In some embodiments, an expression construct further comprises an internal ribosomal entry site (IRES). In some embodiments, an IRES is located between a first gene product and a second gene product.

In some embodiments, an expression construct further comprises a self-cleaving peptide coding sequence. In some embodiments, a self-cleaving peptide is a T2A peptide.

In some embodiments, an expression construct comprises two adeno-associated virus (AAV) inverted terminal repeat (ITR) sequences. In some embodiments. ITR sequences flank a first gene product and a second gene product (e.g., are arranged as follows from 5′-end to 3′-end: ITR-first gene product-second gene product-ITR). In some embodiments, one of the ITR sequences of an isolated nucleic acid lacks a functional terminal resolution site (trs). For example, in some embodiments, one of the ITRs is a ΔITR.

The disclosure relates, in some aspects, to rAAV vectors comprising an ITR having a modified “D” region (e.g., a D sequence that is modified relative to wild-type AAV2 ITR, SEQ ID NO: 16). In some embodiments, the ITR having the modified D region is the 5′ ITR of the rAAV vector. In some embodiments, a modified “D” region comprises an “S” sequence, for example as set forth in SEQ ID NO: 13. In some embodiments, the ITR having the modified “D” region is the 3′ ITR of the rAAV vector. In some embodiments, a modified “D” region comprises a 3′ITR in which the “D” region is positioned at the 3′ end of the ITR (e.g., on the outside or terminal end of the ITR relative to the transgene insert of the vector). In some embodiments, a modified “D” region comprises a sequence as set forth in SEQ ID NO: 13 or 14.

In some embodiments, an isolated nucleic acid (e.g., an rAAV vector) comprises a TRY region. In some embodiments, a TRY region comprises the sequence set forth in SEQ ID NO: 16.

In some embodiments, an isolated nucleic acid described by the disclosure comprises or consists of the sequence set forth in SEQ ID NO: 1-67.

In some aspects, the disclosure provides a vector comprising an isolated nucleic acid as described by the disclosure. In some embodiments, a vector is a plasmid, or a viral vector. In some embodiments, a viral vector is a recombinant AAV (rAAV) vector or a Baculovirus vector. In some embodiments, an rAAV vector is single-stranded (e.g., single-stranded DNA).

In some aspects, the disclosure provides a host cell comprising an isolated nucleic acid as described by the disclosure or a vector as described by the disclosure.

In some aspects, the disclosure provides a recombinant adeno-associated virus (rAAV) comprising a capsid protein and an isolated nucleic acid or a vector as described by the disclosure.

In some embodiments, a capsid protein is capable of crossing the blood-brain barrier, for example an AAV9 capsid protein or an AAVrh.10 capsid protein. In some embodiments, an rAAV transduces neuronal cells and non-neuronal cells of the central nervous system (CNS).

In some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

In some embodiments, administration comprises direct injection to the CNS of a subject. In some embodiments, direct injection is intracerebral injection, intraparenchymal injection. intrathecal injection, intra-cistema magna injection, or any combination thereof. In some embodiments, direct injection to the CNS of a subject comprises convection enhanced delivery (CED).

In some embodiments, administration comprises peripheral injection. In some embodiments, peripheral injection is intravenous injection.

### BRIEF DESCRIPTION OF THE DRAWINGS

FIG. 1 is a schematic depicting one embodiment of a vector comprising an expression construct encoding Gcase (e.g., GBA1 or a portion thereof) and an inhibitory RNA targeting SCNA.

FIG. 2 is a schematic depicting one embodiment of a plasmid comprising an rAAV vector that includes an expression construct encoding SCNA.

FIG. 3 is a schematic depicting one embodiment of a plasmid comprising an rAAV vector that includes an expression construct encoding an inhibitory RNA targeting SCNA. The inhibitory RNA is positioned within an intron between the promoter sequence and the Gcase encoding sequence.

FIG. 4 is a schematic depicting one embodiment of a plasmid comprising an rAAV vector that includes an expression construct encoding progranulin (PGRN) and an inhibitory RNA targeting SCNA. The inhibitory RNA is positioned within an intron between the promoter sequence and the Gcase encoding sequence.

FIG. 5 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 6 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 7 is a schematic depicting one embodiment of a plasmid comprising an rAAV vector that includes an expression construct encoding Gcase (GBA1) and an inhibitory RNA targeting SCNA. The “D” sequence of the 3′ITR is positioned on the “outside” of the vector.

FIG. 8 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 9 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 10 is a schematic depicting one embodiment of a plasmid comprising an rAAV vector that includes an expression construct encoding Gcase (GBA1) and an inhibitory RNA targeting SCNA.

FIG. 11 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 12 is a schematic depicting one embodiment of a plasmid comprising an rAAV vector that includes an expression construct encoding Gcase (GBA1) and an inhibitory RNA targeting SCNA. The inhibitory RNA is positioned within an intron between the promoter sequence and the Gcase encoding sequence.

FIG. 13 is a schematic depicting one embodiment of a plasmid comprising an rAAV vector that includes an expression construct encoding Gcase (GBA1) and progranulin (PGRN), and an inhibitory RNA targeting TMEM106B. The inhibitory RNA is positioned within an intron between the promoter sequence and the Gcase encoding sequence.

FIG. 14 shows representative data indicating successful silencing of SCNA in vitro by GFP reporter assay (top) and α-Syn assay (bottom).

FIG. 15 shows representative data indicating successful silencing of TMEM106B in vitro by GFP reporter assay (top) and α-Syn assay (bottom).

FIG. 16 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIG. 17 shows data for transduction of HEK293 cells using rAA Vs having ITRs with wild-type (circles) or alternative (e.g., “outside”: squares) placement of the “D” sequence. The rAAVs having ITRs placed on the “outside” were able to transduce cells as efficiently as rAAVs having wild-type ITRs.

FIG. 18 is a schematic depicting one embodiment of a plasmid comprising an rAAV vector that includes an expression construct encoding an inhibitory RNA targeting RPS25.

FIG. 19 is a schematic depicting one embodiment of a plasmid comprising an rAAV vector that includes an expression construct encoding an inhibitory RNA targeting RPS25.

FIG. 20 is a schematic depicting one embodiment of a plasmid comprising an rAAV vector that includes an expression construct encoding an inhibitory RNA targeting MAPT.

FIG. 21 is a schematic depicting one embodiment of a plasmid comprising an rAAV vector that includes an expression construct encoding an inhibitory RNA targeting MAPT.

FIG. 22 is a schematic depicting one embodiment of a plasmid comprising an rAAV vector that includes an expression construct encoding progranulin (PGRN) and an inhibitory RNA targeting MAPT. The inhibitory RNA is positioned within an intron between the promoter sequence and the PGRN encoding sequence.

FIG. 23 is a schematic depicting one embodiment of a plasmid comprising an rAAV vector that includes an expression construct encoding an inhibitory RNA targeting MAPT.

FIG. 24 is a schematic depicting one embodiment of a plasmid comprising an rAAV vector that includes an expression construct encoding progranulin (PGRN) and an inhibitory RNA targeting MAPT. The inhibitory RNA is positioned within an intron between the promoter sequence and the PGRN encoding sequence.

### DETAILED DESCRIPTION

The disclosure is based, in part, on compositions and methods for expression of combinations of PD-associated gene products in a subject. A gene product can be a protein, a fragment (e.g., portion) of a protein, an interfering nucleic acid that inhibits a PD-associated gene, etc. In some embodiments, a gene product is a protein or a protein fragment encoded by a PD-associated gene. In some embodiments, a gene product is an interfering nucleic acid (e.g., shRNA, siRNA, miRNA, amiRNA, etc.) that inhibits a PD-associated gene.

A PD-associated gene refers to a gene encoding a gene product that is genetically, biochemically or functionally associated with PD. For example, individuals having mutations in the GBA1 gene (which encodes the protein Gcase), have been observed to be have an increased risk of developing PD compared to individuals that do not have a mutation in GBA1. In another example, PD is associated with accumulation of protein aggregates comprising α-Synuclein (α-Syn) protein; accordingly, SCNA (which encodes α-Syn) is a PD-associated gene. In some embodiments, an expression cassette described herein encodes a wild-type or non-mutant form of a PD-associated gene (or coding sequence thereof). Examples of PD-associated genes are listed in Table 1.

TABLE 1
Examples of PD-associated genes

| | | | NCBI Accession |
|---|---|---|---|
| Name | Gene | Function | No. |
| alpha-Synuclein | SNCA | plays a role in | NP_001139527.1 |
| | | maintaining a supply | |
| | | of synaptic vesicles | |
| | | in presynaptic | |
| | | terminals by | |
| | | clustering synaptic | |
| | | vesicles, and may | |
| | | help regulate the | |
| | | release of dopamine | |
| beta-Glucocerebrosidase | GBA1 | cleaves the beta- | NP_001005742.1 |
| | | glucosidic linkage of | (Isoform 1), |
| | | glucocerebroside | NP_001165282.1 |
| | | | (Isoform 2), |
| | | | NP_001165283.1 |
| | | | (Isoform 3) |
| Transmembrane | TMEM106B | plays a role | NP_060844.2 |
| protein 106B | | in dendrite | |
| | | morphogenesis | |
| | | and regulation | |
| | | of lysosomal | |
| | | trafficking | |
| Progranulin | PGRN | plays a role in | NP_002087.1 |
| | | development, | |
| | | inflammation, cell | |
| | | proliferation and | |
| | | protein homeostasis | |
| Ribosomal protein S25 | RPS25 | ribosomal protein | AB061844.1 |
| | | that is a component | |
| | | of the 40S subunit | |
| Microtubule-associated | MAPT | Microtubule | NM_016835.4 |
| protein tau | | stabilization | |

### Isolated Nucleic Acids and Vectors

An isolated nucleic acid may be DNA or RNA. In some aspects, the disclosure provides isolated nucleic acids (e.g., rAAV vectors) encoding one or more inhibitory nucleic acids that target one or more PD-associated gene, for example SCNA, TMEM 106B, RPS25, and MAPT. In some embodiments, the isolated nucleic acids further comprise a protein-encoding sequence, for example a nucleic acid sequence encoding a Gcase (e.g., GBA1) or progranulin (e.g., PGRN).

Generally, an isolated nucleic acid as described herein may encode 1, 2, 3, 4, 5, 6, 7, 8, 9, 10, or more inhibitory nucleic acids (e.g., dsRNA, siRNA, shRNA, miRNA, amiRNA, etc.). In some embodiments, an isolated nucleic acid encodes more than 10 inhibitory nucleic acids. In some embodiments, each of the one or more inhibitory nucleic acids targets a different gene or a portion of a gene (e.g., a first miRNA targets a first target sequence of a gene and a second miRNA targets a second target sequence of the gene that is different than the first target sequence). In some embodiments, each of the one or more inhibitory nucleic acids targets the same target sequence of the same gene (e.g., an isolated nucleic acid encodes multiple copies of the same miRNA).

Aspects of the disclosure relate to an isolated nucleic acid comprising an expression construct encoding one or more interfering nucleic acids (e.g., dsRNA, siRNA, miRNA, amiRNA, etc.) that target an α-Synuclein protein (e.g., the gene product of SCNA gene). α-Synuclein protein refers to a protein found in brain tissue, which is plays a role in maintaining a supply of synaptic vesicles in presynaptic terminals by clustering synaptic vesicles and regulating the release of dopamine. In humans, SCNA gene is located on chromosome 4. In some embodiments, the SCNA gene encodes a peptide that is represented by NCBI Reference Sequence NP_001139527.1. In some embodiments, a SCNA gene comprises the sequence set forth in SEQ ID NO: 1.

An inhibitory nucleic acid targeting SCNA may comprise a region of complementarity (e.g., a region of the inhibitory nucleic acid that hybridizes to the target gene, such as SCNA) that is between 6 and 50 nucleotides in length. In some embodiments, an inhibitory nucleic acid comprises a region of complementarity with SCNA that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a SCNA sequence.

Aspects of the disclosure relate to an isolated nucleic acid comprising an expression construct encoding one or more interfering nucleic acids (e.g., dsRNA, siRNA, miRNA. amiRNA, etc.) that target an TMEM106B protein (e.g., the gene product of SCNA gene). TMEM 106B protein refers to transmembrane protein 106B, which is a protein involved in dendrite morphogenesis and regulation of lysosomal trafficking. In humans, TMEM106B gene is located on chromosome 7. In some embodiments, the TMEM106B gene encodes a peptide that is represented by NCBI Reference Sequence NP_060844.2. In some embodiments, a TMEM 106B gene comprises the sequence set forth in SEQ ID NO: 2.

An inhibitory nucleic acid targeting TMEM106B may comprise a region of complementarity (e.g., a region of the inhibitory nucleic acid that hybridizes to the target gene, such as TMEM106B) that is between 6 and 50 nucleotides in length. In some embodiments, an inhibitory nucleic acid comprises a region of complementarity with TMEM106B that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a TMEM106B sequence.

Aspects of the disclosure relate to an isolated nucleic acid comprising an expression construct encoding one or more interfering nucleic acids (e.g., dsRNA, siRNA, miRNA, amiRNA, etc.) that target an ribosomal protein s25 (RPS25) (e.g., the gene product of RPS25). RPS25 protein refers to a ribosomal protein which is a subunit of the s40 ribosome, a protein complex involved in protein synthesis. In humans, RPS25 gene is located on chromosome 11. In some embodiments, the RPS25 gene encodes a peptide that is represented by NCBI Reference Sequence NP_001019.1. In some embodiments, a RPS25 gene comprises the sequence set forth in SEQ ID NO: 36.

An inhibitory nucleic acid targeting RPS25 may comprise a region of complementarity (e.g., a region of the inhibitory nucleic acid that hybridizes to the target gene, such as RPS25) that is between 6 and 50 nucleotides in length. In some embodiments, an inhibitory nucleic acid comprises a region of complementarity with RPS25 that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a RPS25 sequence.

Aspects of the disclosure relate to an isolated nucleic acid comprising an expression construct encoding one or more interfering nucleic acids (e.g., dsRNA, siRN, miRNA, amiRNA, etc.) that target an microtubule-associated protein tau, MAPT (e.g., the gene product of MAPT gene). MAPT protein refers to microtubule-associated protein tau, which is a protein involved in microtubule stabilization. In humans, MAPT gene is located on chromosome 17. In some embodiments, the MAPT gene encodes a peptide that is represented by NCBI Reference Sequence NP_005901.2. In some embodiments, a MAPT gene comprises the sequence set forth in SEQ ID NO: 37.

An inhibitory nucleic acid targeting MAPT may comprise a region of complementarity (e.g., a region of the inhibitory nucleic acid that hybridizes to the target gene, such as MAPT) that is between 6 and 50 nucleotides in length. In some embodiments, an inhibitory nucleic acid comprises a region of complementarity with MAPT that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a MAPT sequence.

In some aspects, the disclosure provides an isolated nucleic acid comprising an expression construct encoding a first gene product and a second gene product, wherein each gene product independently is selected from the gene products, or portions thereof, set forth in Table 1.

In some embodiments, a gene product is encoded by a coding portion (e.g., a cDNA) of a naturally occurring gene. In some embodiments, a first gene product is a protein (or a fragment thereof) encoded by the GBA1 gene. In some embodiments, a gene product is an inhibitory nucleic acid that targets (e.g., hybridizes to, or comprises a region of complementarity with) a PD-associated gene (e.g., SCNA). A skilled artisan recognizes that the order of expression of a first gene product (e.g., Gcase) and a second gene product (e.g., inhibitory RNA targeting SCNA) can generally be reversed (e.g., the inhibitory RNA is the first gene product and Gcase is the second gene product). In some embodiments, a gene product is a fragment (e.g., portion) of a gene listed in Table 1. A protein fragment may comprise about 50%, about 60%, about 70%, about 80% about 90% or about 99% of a protein encoded by the genes listed in Table 1. In some embodiments, a protein fragment comprises between 50% and 99.9% (e.g., any value between 50% and 99.9%) of a protein encoded by a gene listed in Table 1. In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example SCNA).

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornøe et al. (2002) Gene 297 (1-2): 21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34(Database issue): D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7:2193. In some embodiments, the self-cleaving peptide is a T2A peptide.

Pathologically, disorders such as PD and Gaucher disease are associated with accumulation of protein aggregates composed largely of α-Synuclein (α-Syn) protein. Accordingly, in some embodiments, isolated nucleic acids described herein comprise an inhibitory nucleic acid that reduces or prevents expression of α-Syn protein. A sequence encoding an inhibitory nucleic acid may be placed in an untranslated region (e.g., intron, 5′UTR. 3′UTR, etc.) of the expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), siRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets SNCA (the gene encoding α-Syn protein). In some embodiments, the miRNA does not comprise any mismatches with the region of SNCA mRNA to which it hybridizes (e.g., the miRNA is “perfected”). In some embodiments, the inhibitory nucleic acid is an shRNA (e.g., an shRNA targeting SNCA).

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting SCNA or TMEM106B.

In some embodiments, an inhibitory nucleic acid targeting SCNA comprises a miRNA/miRNA* duplex. In some embodiments, the miRNA strand of a miRNA/miRNA* duplex comprises or consists of the sequence set forth in any one of SEQ ID NOs: 3-8. In some embodiments, the miRNA* strand of a miRNA/miRNA* duplex comprises or consists of the sequence set forth in any one of SEQ ID NOs: 3-8

In some embodiments, an inhibitory nucleic acid targeting TMEM106B comprises a miRNA/miRNA* duplex. In some embodiments, the miRNA strand of a miRNA/miRNA* duplex comprises or consists of the sequence set forth in SEQ ID NO: 9 or 10. In some embodiments, the miRNA* strand of a miRNA/miRNA* duplex comprises or consists of the sequence set forth in SEQ ID NOs: 9 or 10.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, scAAV vectors and scAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99. pri-MIR-194, pri-MIR-155, and pri-MIR-451. In some embodiments, an amiRNA comprises a nucleic acid sequence targeting SCNA or TMEM106B and an cSIBR amiRNA scaffold, for example as described in Fowler et al. Nucleic Acids Res. 2016 Mar. 18; 44 (5): e48.

In some embodiments, an amiRNA targeting SCNA comprises or consists of the sequence set forth in any one of SEQ ID NOs: 17-22. In some embodiments, an amiRNA targeting TMEM106B comprises or consists of the sequence set forth in SEQ ID NOs: 11 or 12. In some embodiments, an amiRNA targeting RPS25 comprises or consists of the sequence set forth in SEQ ID NOs: 38 to 45. In some embodiments, an amiRNA targeting MAPT comprises or consists of the sequence set forth in SEQ ID NOs: 46 to 61.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculovirus vector, etc.). In some embodiments, the vector is a plasmid (e.g., a plasmid comprising an isolated nucleic acid as described herein). In some embodiments, the vector is a recombinant AAV (rAAV) vector. In some embodiments, an rAAV vector is single-stranded (e.g., single-stranded DNA). In some embodiments, a vector is a Baculovirus vector (e.g., an Autographa californica nuclear polyhedrosis (AcNPV) vector).

Typically an rAAV vector (e.g., rAAV genomc) comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a ΔITR, for example as described by McCarty et al. (2003) Gene Ther. 10(26): 2112-8.

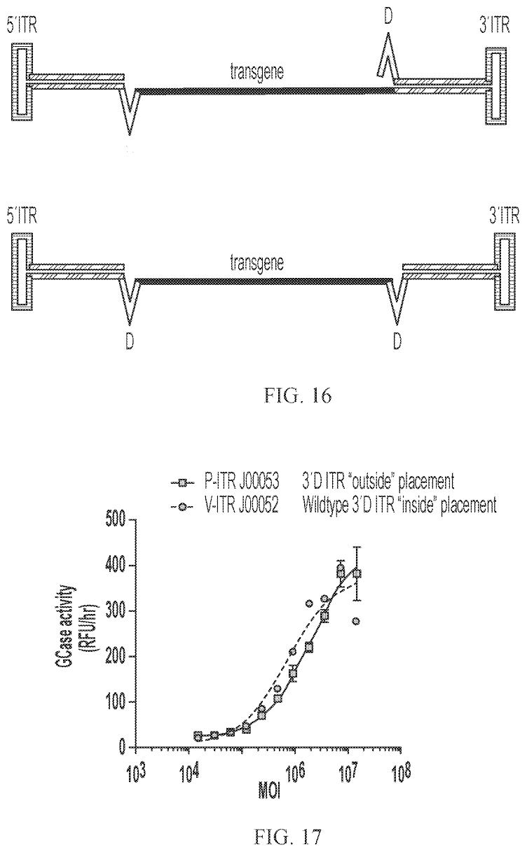

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 16). The structure of wild-type AAV2 ITR is shown in FIG. 16. Generally, a wild-type ITR comprises a 125 nucleotide region that self-anneals to form a palindromic double-stranded T-shaped, hairpin structure consisting of two cross arms (formed by sequences referred to as B/B′ and C/C′. respectively), a longer stem region (formed by sequences A/A′), and a single-stranded terminal region referred to as the “D” region. (FIG. 16). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 14. The “D” region has been observed to play an important role in encapsidation of rAAV vectors by capsid proteins, for example as disclosed by Ling et al. (2015) J Mol Genet Med 9(3).

The disclosure is based, in part, on the surprising discovery that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 14). A modified “D” sequence may have at least 1, 2, 3, 4, 5, 6, 7, 8, 9, 10, or more than 10 nucleotide substitutions relative to a wild-type “D” sequence (e.g., SEQ ID NO: 14). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 13). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 14). In some embodiments, a modified “D” sequence comprises the sequence set forth in SEQ ID NO: 13, also referred to as an “S” sequence as described in Wang et al. (1995) J Mol Biol 250(5): 573-80.

An isolated nucleic acid or rAAV vector as described by the disclosure may further comprise a “TRY” sequence, for example as set forth in SEQ ID NO: 15, as described by Francois, et al. 2005. The Cellular TATA Binding Protein Is Required for Rep-Dependent Replication of a Minimal Adeno-Associated Virus Type 2 p5 Element. J Virol. In some embodiments, a TRY sequence is positioned between an ITR (e.g., a 5′ ITR) and an expression construct (e.g., a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13 (16): 1935-43 and Smith et al. (2009) Mol Ther 17 (11): 1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

### rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20 25, 50, 100, etc.) amino acid substitutions (e.g., mutations) relative to the wild-type AAV capsid protein from which it is derived.

In some embodiments rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63 (9): 3822-8 and Wright (2009) Hum Gene Ther. 20 (7): 698-706.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13 (16): 1935-43, Smith et al. (2009) Mol Ther 17 (11): 1888-1896, U.S. Pat. Nos. 8,945,918, 9,879,282, and International PCT Publication WO 2017/184879. However, an rAAV can be produced using any suitable method (e.g., using recombinant rep and cap genes).

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

### Methods

The disclosure is based, in part, on compositions for expression of combinations of PD-associated gene products in a subject that act together (e.g., synergistically) to treat Parkinson's disease. As used herein “treat” or “treating” refers to (a) preventing or delaying onset of Parkinson's disease: (b) reducing severity of Parkinson's disease; (c) reducing or preventing development of symptoms characteristic of Parkinson's disease; (d) and/or preventing worsening of symptoms characteristic of Parkinson's disease. Symptoms of Parkinson's disease include, for example, motor dysfunction (e.g., shaking, rigidity, slowness of movement, difficulty with walking), cognitive dysfunction (e.g., dementia, depression, anxiety), emotional and behavioral dysfunction.

Accordingly, in some aspects, the disclosure provides a method for treating a subject having or suspected of having Parkinson's disease, the method comprising administering to the subject a composition (e.g., a composition comprising an isolated nucleic acid or a vector or a rAAV) as described by the disclosure.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9 (10): 1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Example 1: rAAV Vectors

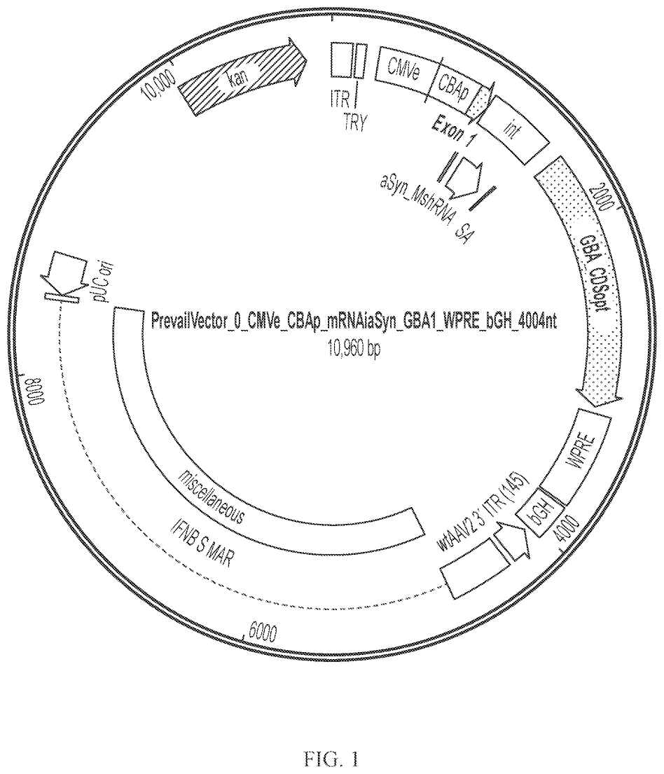

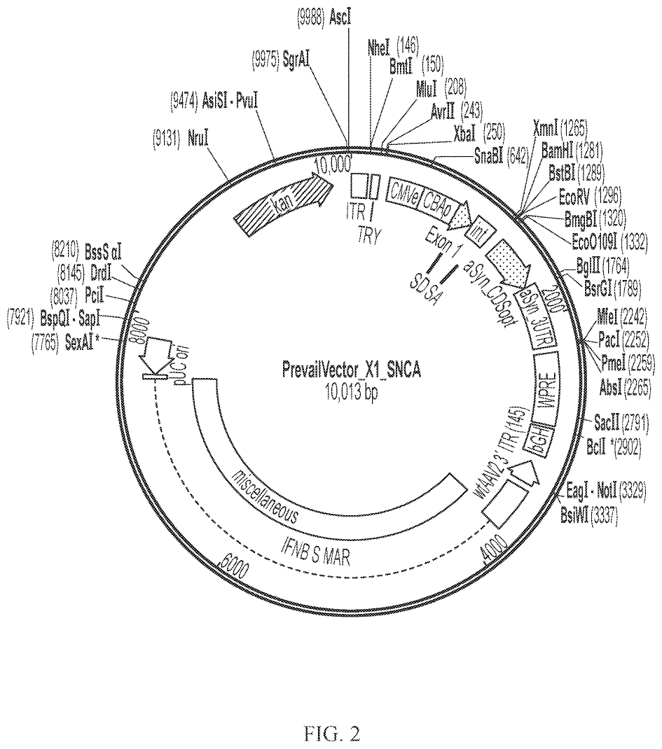

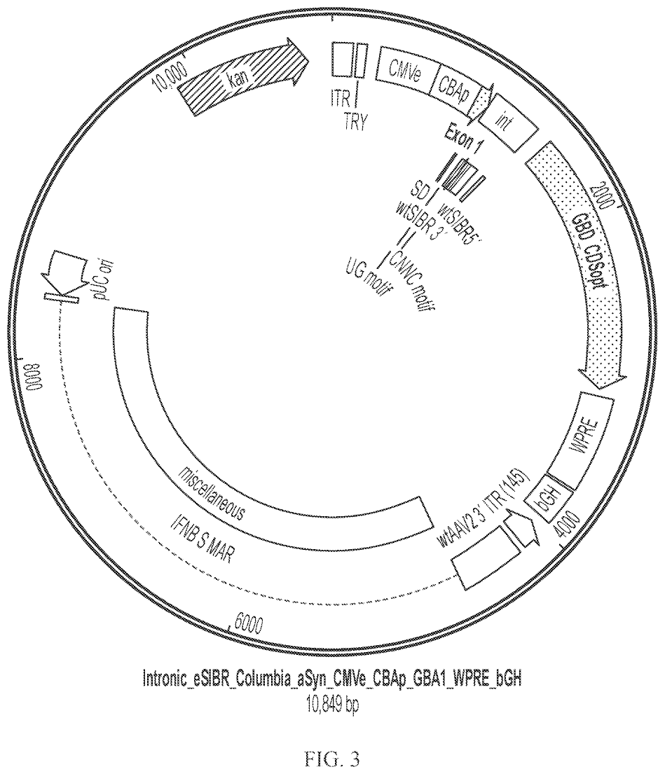

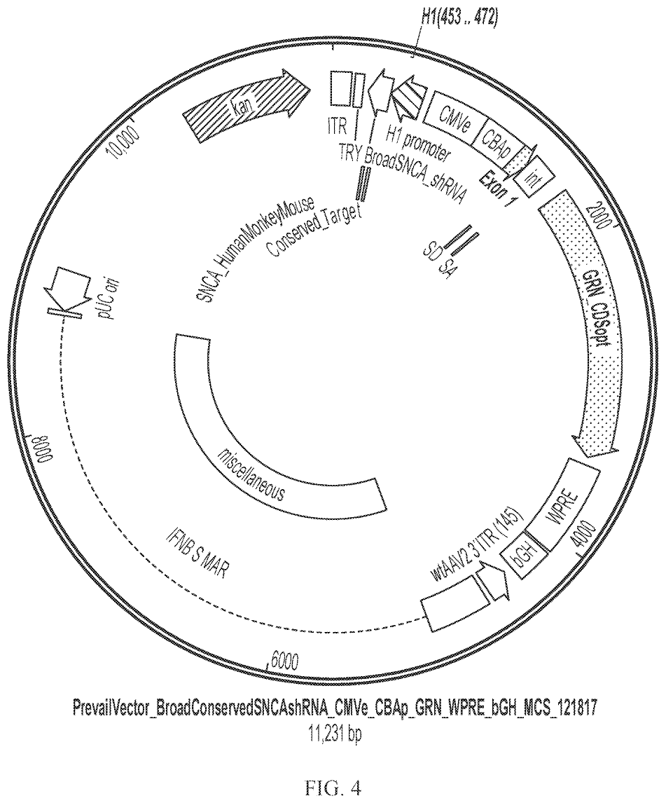

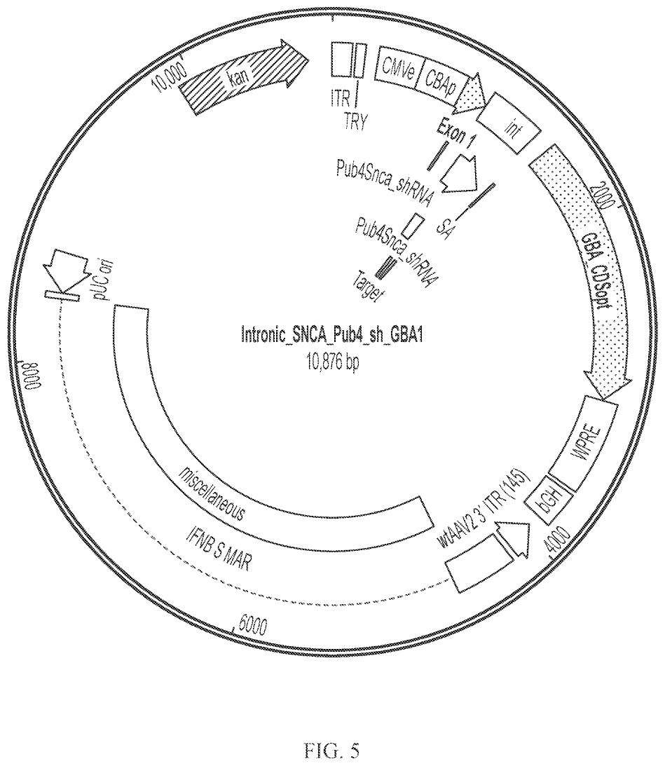

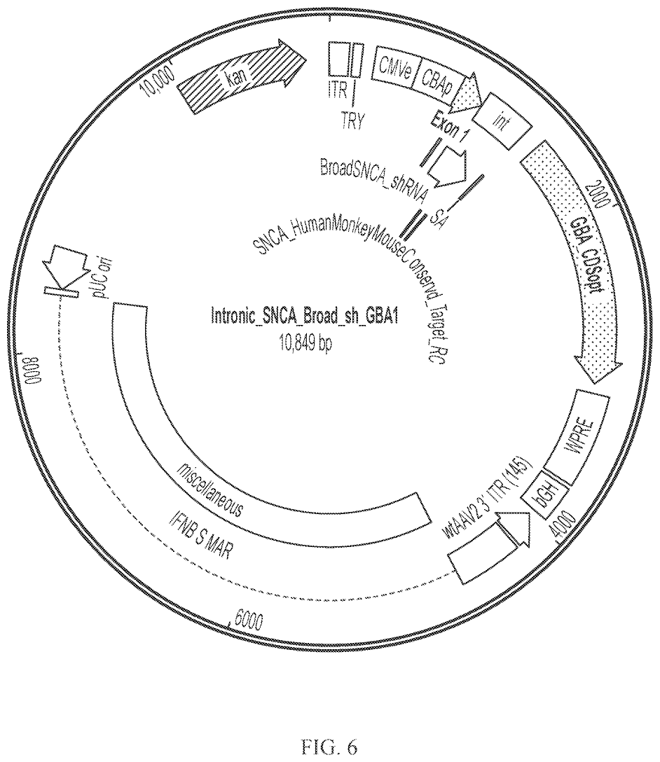

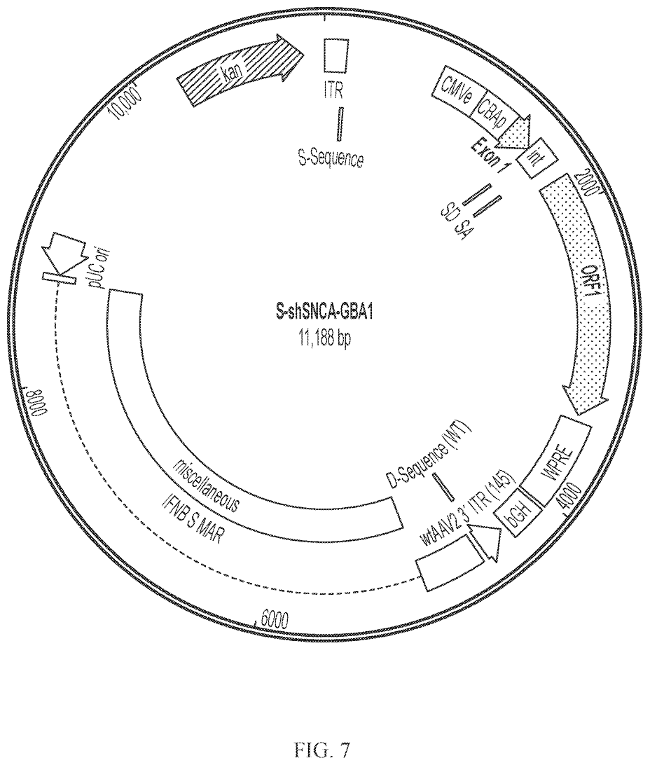

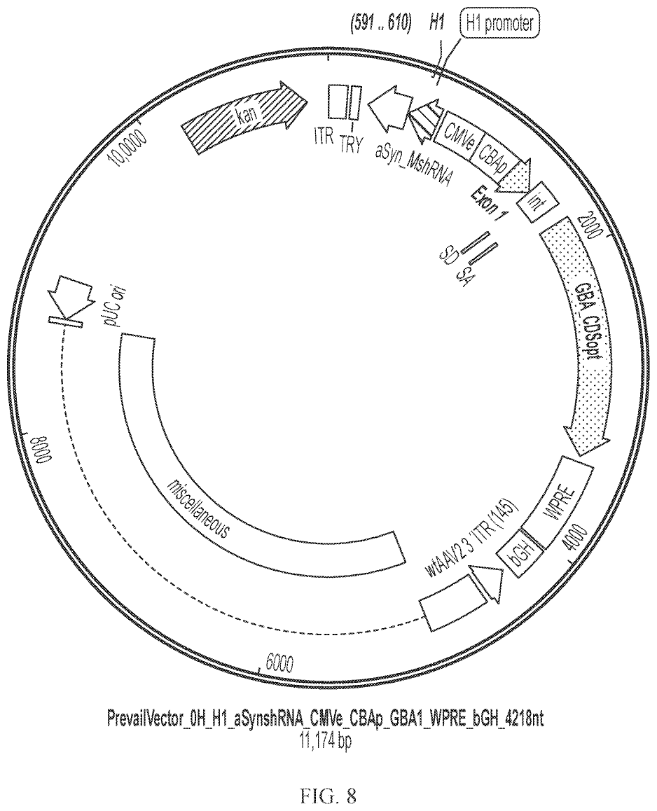

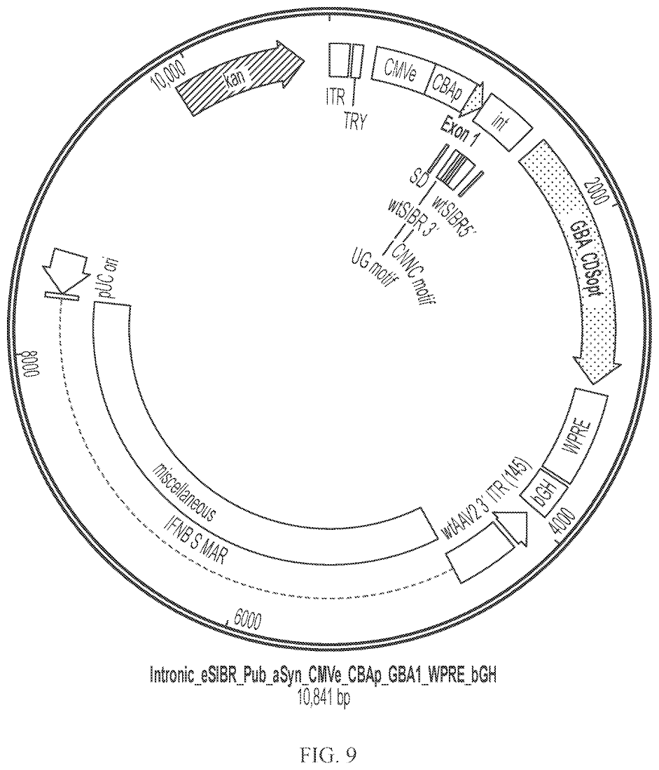

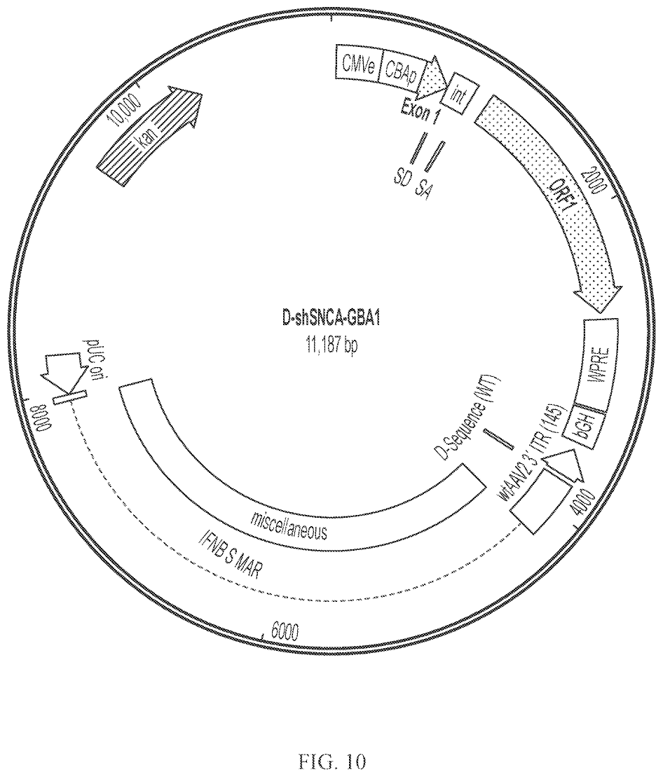

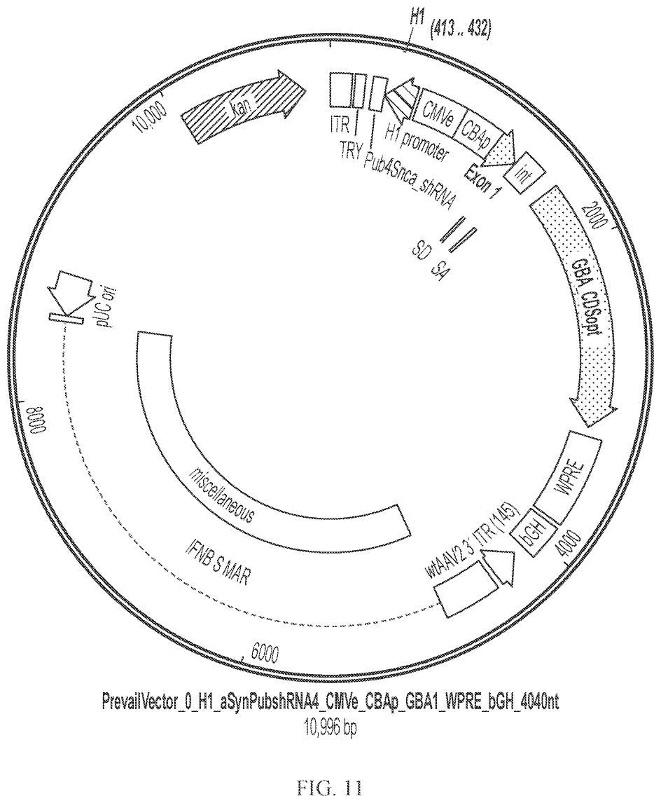

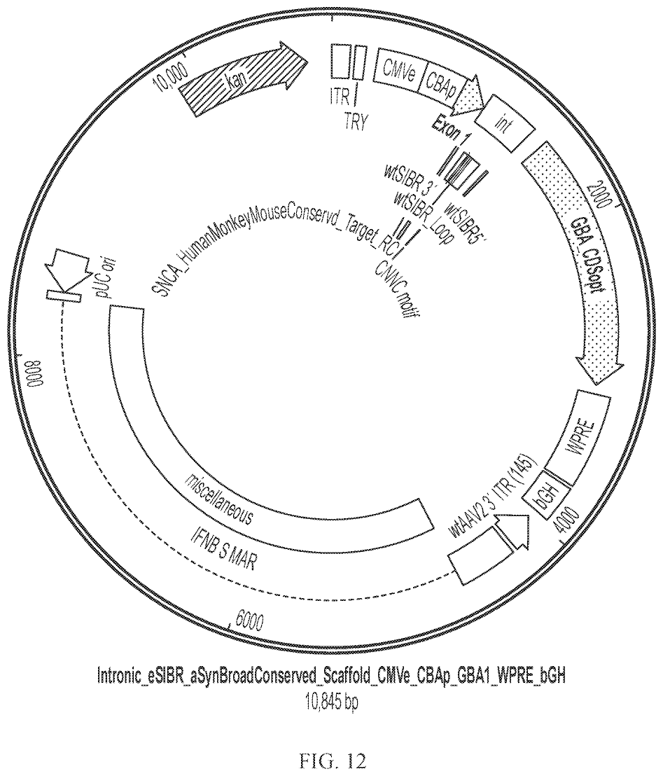

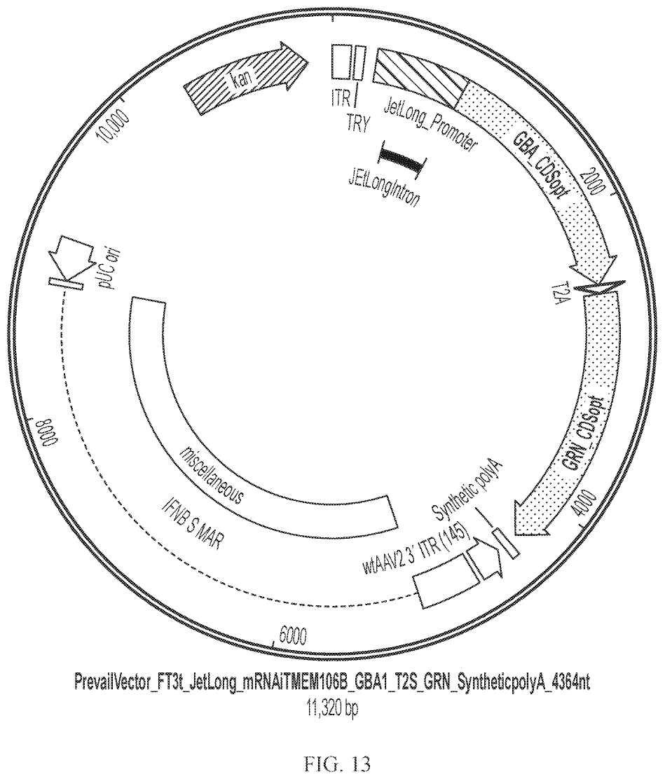

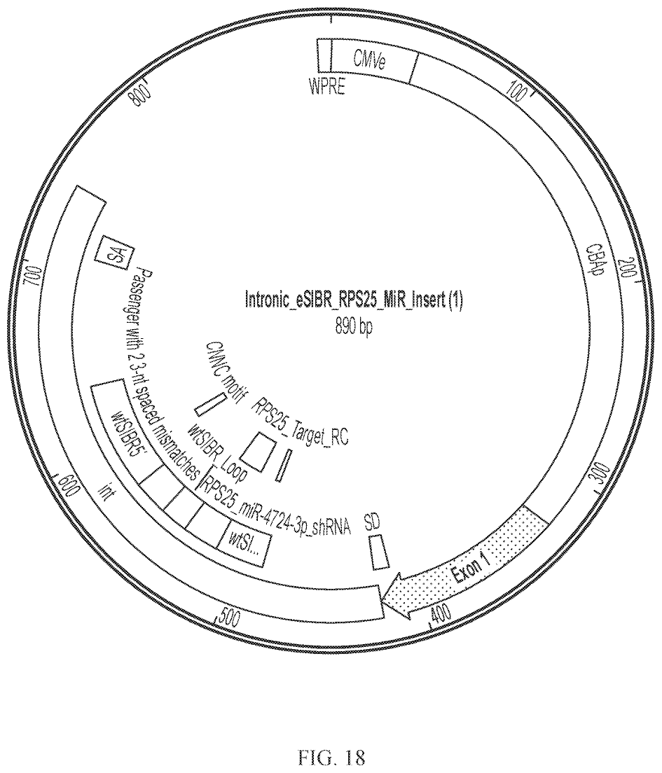

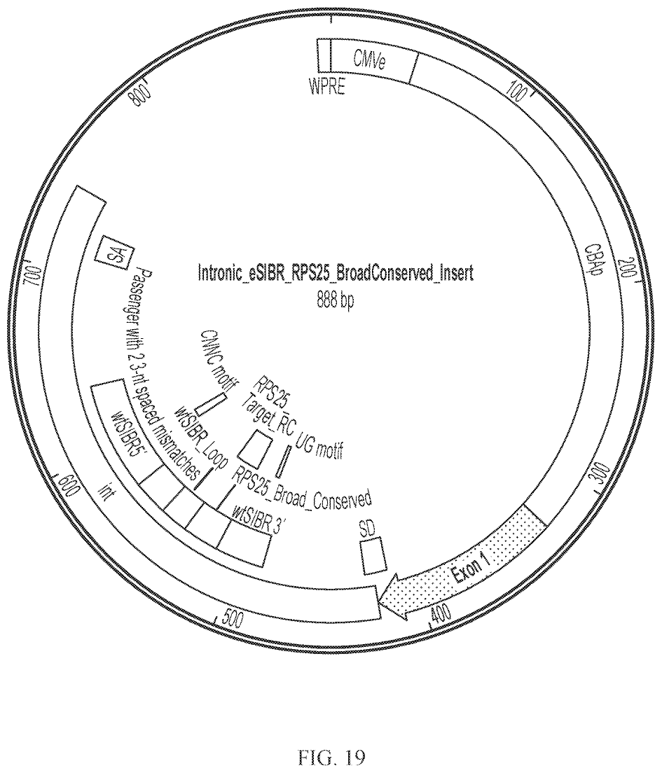

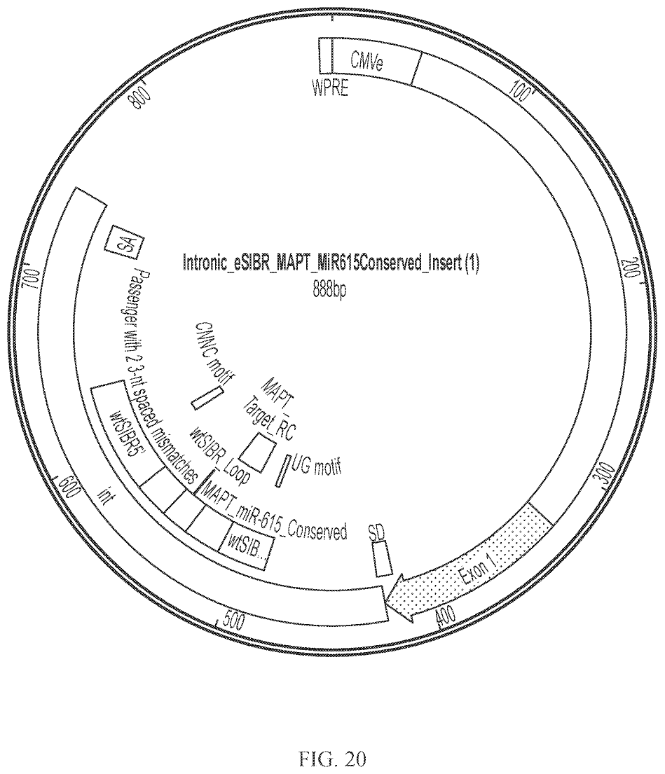

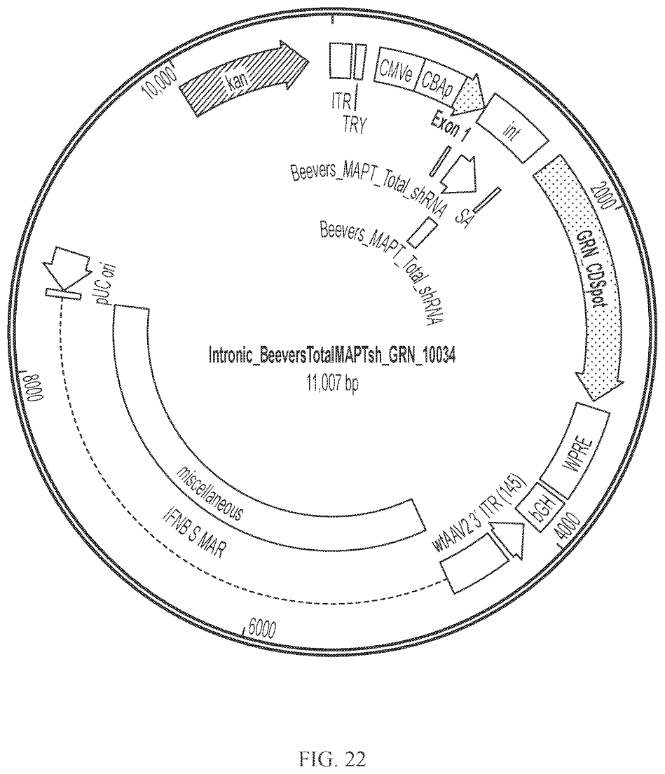

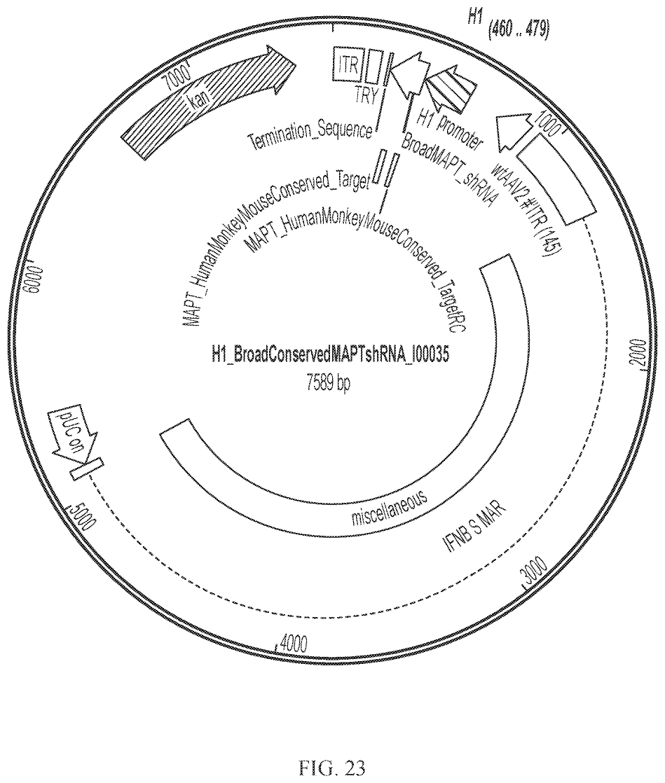

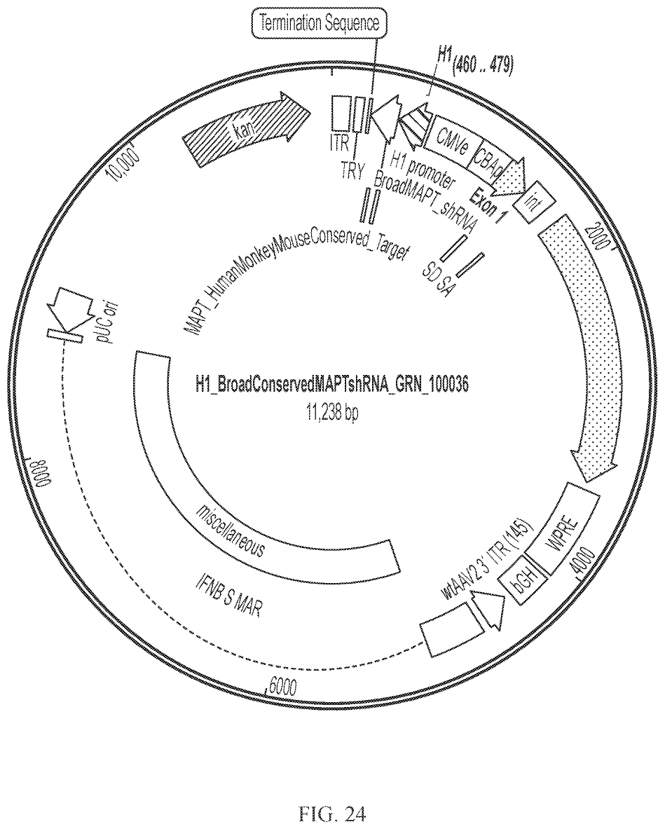

AAV vectors are generated using cells, such as HEK293 cells for triple-plasmid transfection. The ITR sequences flank an expression construct comprising a promoter/enhancer element for each transgene of interest, a 3′ polyA signal, and posttranslational signals such as the WPRE element. Multiple gene products can be expressed simultaneously such as GBA1 and one or more inhibitory nucleic acids (e.g., inhibitory nucleic acids targeting SCNA), for example by expression with 2 separate expression cassettes. The presence of a short intronic sequence that is efficiently spliced, upstream of the expressed gene, can improve expression levels. shRNAs and other regulatory RNAs can potentially be included within these sequences. Examples of expression constructs described by the disclosure are shown in FIGS. 1-13 and 18-24, and in Table 2 below.

TABLE 2

| | | | | | | | | | Length |
|---|---|---|---|---|---|---|---|---|---|
| | Promoter | | | | Bicistronic | Promotor | | | between |
| Name | 1 | shRNA | CDS1 | PolyA1 | element | 2 | CDS2 | PolyA2 | ITRs |
| PrevailVector_0_CMVe_CBAp_mRNA- | CBA | aSyn | GBA1 | WPRE_bGH | — | — | — | — | 4004 |
| iaSyn_GBA1_WPRE_bGH_4004nt | | | | | | | | | |
| PrevailVector_Xl_SNCA | CMVe + | — | SNCA | WPRE_bGH | — | — | — | — | — |
| | CBA | | | | | | | | |

### Example 2: Cell Based Assays of Viral Transduction Into GBA-Deficient Cells

Cells deficient in GBA1 are obtained, for example as fibroblasts from GD patients, monocytes, or hES cells, or patient-derived induced pluripotent stem cells (iPSCs). These cells accumulate substrates such as glucosylceramide and glucosylsphingosine (GluCer and GluSph). Treatment of wild-type or mutant cultured cell lines with Gcase inhibitors, such as CBE, is also be used to obtain GBA deficient cells.

Using such cell models, lysosomal defects are quantified in terms of accumulation of protein aggregates, such as of α-Synuclein with an antibody for this protein or phospho-αSyn. followed by imaging using fluorescent microscopy. Imaging for lysosomal abnormalities by ICC for protein markers such as LAMP1, LAMP2, LIMP1, LIMP2, or using dyes such as Lysotracker, or by uptake through the endocytic compartment of fluorescent dextran or other markers is also performed. Imaging for autophagy marker accumulation due to defective fusion with the lysosome, such as for LC3, can also be performed. Western blotting and/or ELISA is used to quantify abnormal accumulation of these markers. Also, the accumulation of glycolipid substrates and products of GBA1 is measured using standard approaches.

Therapeutic endpoints (e.g., reduction of PD-associated pathology) are measured in the context of expression of transduction of the AAV vectors, to confirm and quantify activity and function. Gcase can is also quantified using protein ELISA measures, or by standard Gcase activity assays.

### Example 3: In Vivo Assays Using Mutant Mice

This example describes in vivo assays of AAV vectors using mutant mice. In vivo studies of AAV vectors as above in mutant mice are performed using assays described, for example, by Liou et al. (2006) J. Biol. Chem. 281 (7): 4242-4253, Sun et al. (2005) J. Lipid Res. 46:2102-2113, and Farfel-Becker et al. (2011) Dis. Model Mech. 4 (6): 746-752.

The intrathecal or intraventricular delivery of vehicle control and AAV vectors (e.g., at a dose of 2×10¹¹ vg/mouse) are performed using concentrated AAV stocks, for example at an injection volume between 5-10 μL. Intraparenchymal delivery by convection enhanced delivery is performed.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 4: Chemical Models of Disease

This example describes in vivo assays of AAV vectors using a chemically-induced mouse model of Gaucher disease (e.g., the CBE mouse model). In vivo studies of these AAV vectors are performed in a chemically-induced mouse model of Gaucher disease, for example as described by Vardi et al. (2016) J Pathol. 239 (4): 496-509.

Intrathecal or intraventricular delivery of vehicle control and AAV vectors (e.g., at a dose of 2×10¹¹ vg/mouse) are performed using concentrated AAV stocks, for example with injection volume between 5-10 μL. Intraparenchymal delivery by convection enhanced delivery is performed. Peripheral delivery is achieved by tail vein injection.

Treatment is initiated either before onset of symptoms, or subsequent to onset. Endpoints measured are the accumulation of substrate in the CNS and CSF, accumulation of Gcase enzyme by ELISA and of enzyme activity, motor and cognitive endpoints, lysosomal dysfunction, and accumulation of α-Synuclein monomers, protofibrils or fibrils.

### Example 5: Clinical Trials in PD, LBD, Gaucher Disease Patients

In some embodiments, patients having certain forms of Gaucher disease (e.g., GD1) have an increased risk of developing Parkinson's disease (PD) or Lewy body dementia (LBD). This Example describes clinical trials to assess the safety and efficacy of rAAVs as described by the disclosure, in patients having Gaucher disease, PD and/or LBD.

Clinical trials of such vectors for treatment of Gaucher disease, PD and/or LBD are performed using a study design similar to that described in Grabowski et al. (1995) Ann. Intern. Med. 122 (1): 33-39.

### Example 6: Treatment of Peripheral Disease

In some embodiments, patients having certain forms of Gaucher disease exhibit symptoms of peripheral neuropathy, for example as described in Biegstraaten et al. (2010) Brain 133 (10): 2909-2919.

This example describes in vivo assays of AAV vectors as described herein for treatment of peripheral neuropathy associated with Gaucher disease (e.g., Type 1 Gaucher disease). Briefly. Type 1 Gaucher disease patients identified as having signs or symptoms of peripheral neuropathy are administered a rAAV as described by the disclosure. In some embodiments, the peripheral neuropathic signs and symptoms of the subject are monitored, for example using methods described in Biegstraaten et al., after administration of the rAAV.

Levels of transduced gene products as described by the disclosure present in patients (e.g., in serum of a patient, in peripheral tissue (e.g., liver tissue, spleen tissue, etc.)) of a patient are assayed, for example by Western blot analysis, enzymatic functional assays, or imaging studies.

### Example 7: Treatment of CNS Forms

This example describes in vivo assays of rAAVs as described herein for treatment of CNS forms of Gaucher disease. Briefly, Gaucher disease patients identified as having a CNS form of Gaucher disease (e.g., Type 2 or Type 3 Gaucher disease) are administered a rAAV as described by the disclosure. Levels of transduced gene products as described by the disclosure present in the CNS of patients (e.g., in serum of the CNS of a patient, in cerebrospinal fluid (CSF) of a patient, or in CNS tissue of a patient) are assayed, for example by Western blot analysis, enzymatic functional assays, or imaging studies.

### Example 8: Testing of SCNA and TMEM106B shRNA Constructs

### HEK293 Cells

Human embryonic kidney 293 cell line (HEK293) were used in this study (#85120602, Sigma-Aldrich). HEK293 cells were maintained in culture media (D-MEM [#11995065, Thermo Fisher Scientific] supplemented with 10% fetal bovine serum [FBS] [#10082147, Thermo Fisher Scientific]) containing 100 units/ml penicillin and 100 μg/ml streptomycin (#15140122, Thermo Fisher Scientific).

### Plasmid Transfection

Plasmid transfection was performed using Lipofectamine 2000 transfection reagent (#11668019. Thermo Fisher Scientific) according to the manufacture's instruction. Briefly, HEK293 cells (#12022001. Sigma-Aldrich) were plated at the density of 3×10⁵ cells/ml in culture media without antibiotics. On the following day, the plasmid and Lipofectamine 2000 reagent were combined in Opti-MEM solution (#31985062, Thermo Fisher Scientific). After 5 minutes, the mixtures were added into the HEK293 culture. After 72 hours, the cells were harvested for RNA or protein extraction, or subjected to the imaging analyses. For imaging analyses, the plates were pre-coated with 0.01% poly-L-Lysine solution (P8920, Sigma-Aldrich) before the plating of cells.

Gene Expression Analysis by Quantitative Real-Time PCR (qRT-PCR)

Relative gene expression levels were determined by quantitative real-time PCR (qRT-PCR) using Power SYBR Green Cells-to-CT Kit (#4402955. Thermo Fisher Scientific) according to the manufacturer's instruction. The candidate plasmids were transiently transfected into HEK293 cells plated on 48-well plates (7.5×10⁴ cells/well) using Lipofectamine 2000 transfection reagent (0.5 μg plasmid and 1.5 μl reagent in 50 μl Opti-MEM solution). After 72 hours, RNA was extracted from the cells and used for reverse transcription to synthesize cDNA according to the manufacturer's instruction. For quantitative PCR analysis, 2˜5 μl of cDNA products were amplified in duplicates using gene specific primer pairs (250 nM final concentration) with Power SYBR Green PCR Master Mix (#4367659, Thermo Fisher Scientific). The primer sequences for SNCA, TMEM 106B, and GAPDH genes were: 5′-AAG AGG GTG TTC TCT ATG TAG GC-3′ (SEQ ID NO: 64), 5′-GCT CCT CCA ACA TTT GTC ACT T-3′ (SEQ ID NO: 65) for SNCA, 5′-ACA CAG TAC CTA CCG TTA TAG CA-3′ (SEQ ID NO: 66), S′-TGT TGT CAC AGT AAC TTG CAT CA-3′ (SEQ ID NO: 67) for TMEM 106B, and 5′-CTG GGC TAC ACT GAG CAC C-3′ (SEQ ID NO: 68), 5′-AAG TGG TCG TTG AGG GCA ATG-3′ (SEQ ID NO: 69) for GAPDH. Quantitative PCR was performed in a QuantStudio 3 Real-Time PCR system (Thermo Fisher Scientific). Expression levels were normalized by the housekeeping gene GAPDH and calculated using the comparative CT method.

### Fluorescence Imaging Analysis

EGFP reporter plasmids, which contain 3′-UTR of human SNCA gene at downstream of EGFP coding region, were used for the validation of SNCA and TMEM106B knockdown plasmids. EGFP reporter plasmids and candidate knockdown plasmids were simultaneously transfected into HEK293 cells plated on poly-L-Lysine coated 96-well plates (3.0×10⁴ cells/well) using Lipofectamine 2000 transfection reagent (0.04 μg reporter plasmid, 0.06 μg knockdown plasmid and 0.3 μl reagent in 10 μl Opti-MEM solution). After 72 hours, the fluorescent intensities of EGFP signal were measured at excitation 488 nm/emission 512 nm using Varioskan LUX multimode reader (Thermo Fisher Scientific). Cells were fixed with 4% PFA at RT for 10 minutes, and incubated with D-PBS containing 40 μg/ml 7-aminoactinomycin D (7-AAD) for 30 min at RT. After washing with D-PBS, the fluorescent intensities of 7-AAD signal were measured at excitation 546 nm/emission 647 nm using Varioskan reader to quantify cell number. Normalized EGFP signal per 7-AAD signal levels were compared with the control knockdown samples.

### Enzyme-Linked Immunmosorbent Assay (ELISA)

α-Synuclein reporter plasmids, which contain 3′-UTR of human SNCA gene or TMEM 106B gene downstream of SNCA coding region, were used for the validation of knockdown plasmids at the protein level. Levels of α-synuclein protein were determined by ELISA (#KHB0061, Thermo Fisher Scientific) using the lysates extracted from HEK293 cells. The candidate plasmids were transiently transfected into HEK293 cells plated on 48-well plates (7.5×10⁴ cells/well) using Lipofectamine 2000 transfection reagent (0.1 μg reporter plasmid, 0.15 μg knockdown plasmid and 0.75 μl reagent in 25 μl Opti-MEM solution). After 72 hours, cells were lysed in radioimmunoprecipitation assay (RIPA) buffer (#89900. Thermo Fisher Scientific) supplemented with protease inhibitor cocktail (#P8340, Sigma-Aldrich), and sonicated for a few seconds. After incubation on ice for 30 min, the lysates were centrifuged at 20,000×g at 4° C. for 15 min, and the supernatant was collected. Protein levels were quantified. Plates were read in a Varioskan plate reader at 450 nm, and concentrations were calculated using SoftMax Pro 5 software. Measured protein concentrations were normalized to total protein concentration determined with a bicinchoninic acid assay (#23225, Thermo Fisher Scientific).

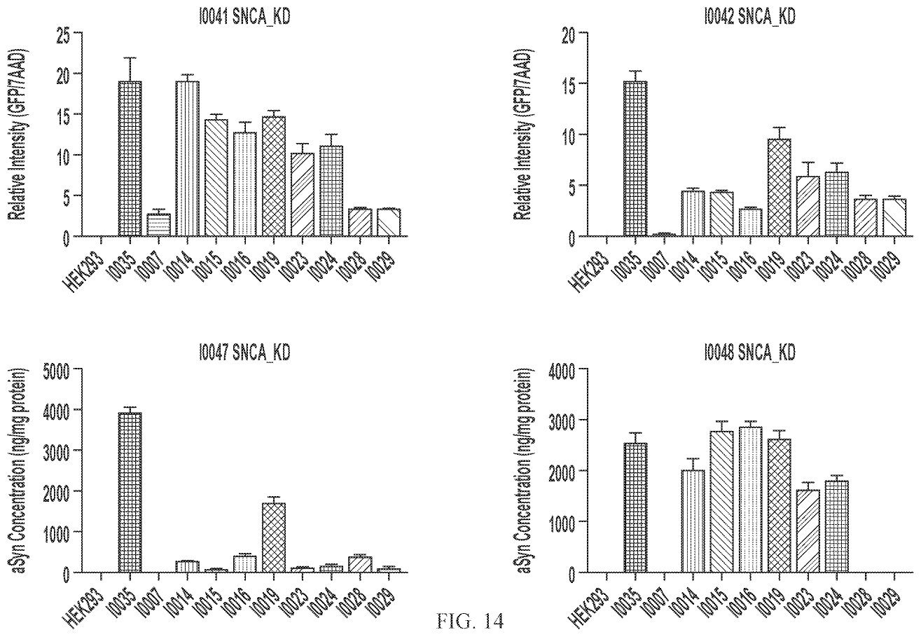

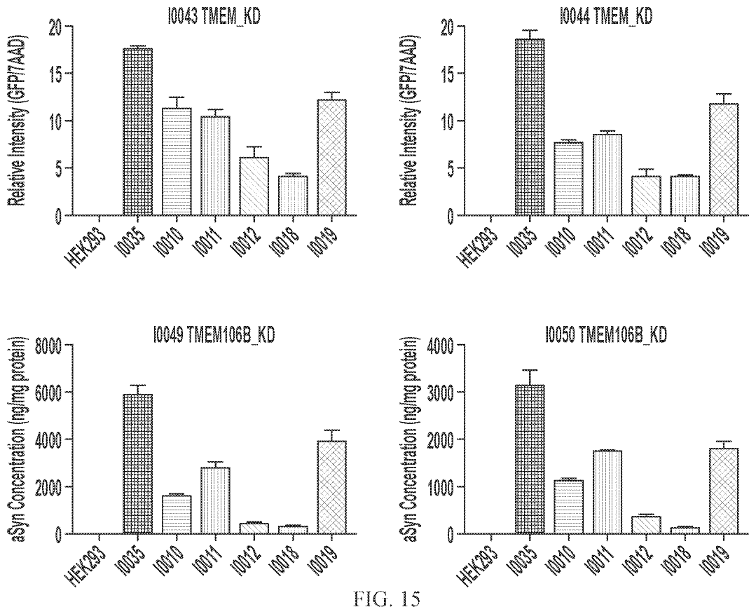

FIG. 14 and Table 3 show representative data indicating successful silencing of SCNA in vitro by GFP reporter assay (top) and α-Syn assay (bottom). FIG. 15 and Table 4 show representative data indicating successful silencing of TMEM106B in vitro by GFP reporter assay (top) and α-Syn assay (bottom).

TABLE 3

| ID | Promoter | Knockdown | Promoter | Overexpress |
|---|---|---|---|---|
| I00007 | CMV_intronic | SNCA_mi | CMV | opt-GBA1 |
| I00008 | H1 | SNCA_sh | CMV | opt-GBA1 |
| I00009 | H1 | SNCA_Pubsh4 | CMV | opt-GBA1 |
| I00014 | JL_intronic | SNCA_mi | JetLong | opt-SCARB2_GBA |
| I00015 | JL_intronic | SNCA_mi | JetLong | opt-PSAP_GBA |
| I00016 | JL_intronic | SNCA_mi | JetLong | opt-CTSB_GBA |
| I00019 | JL_intronic | SNCA_TMEM_mi | JetLong | opt-VPS35 |
| I00023 | JL_intronic | SNCA_mi | JetLong | opt-GBA1_IL34 |
| I00024 | JL_intronic | SNCA_mi | JetLong | opt-GBA2 |
| I00028 | intronic | SNCA_Broadsh | CMV | opt-GBA1 |
| I00029 | intronic | SNCA_Pubsh4 | CMV | opt-GBA1 |

TABLE 4

| ID | Promoter | Knockdown | Promoter | Overexpress |
|---|---|---|---|---|
| I00010 | H1 | TMEM_Pubsh | CMV | opt-GRN |
| I00011 | JL_intronic | TMEM_mi | JetLong | opt-GBA1_GRN |
| I00012 | H1 | TMEM_sh | CMV | opt-GRN |
| I00019 | JL_intronic | SNCA_TMEM_mi | JetLong | opt-VPS35 |

### Example 9: ITR “D” Sequence Placement and Cell Transduction

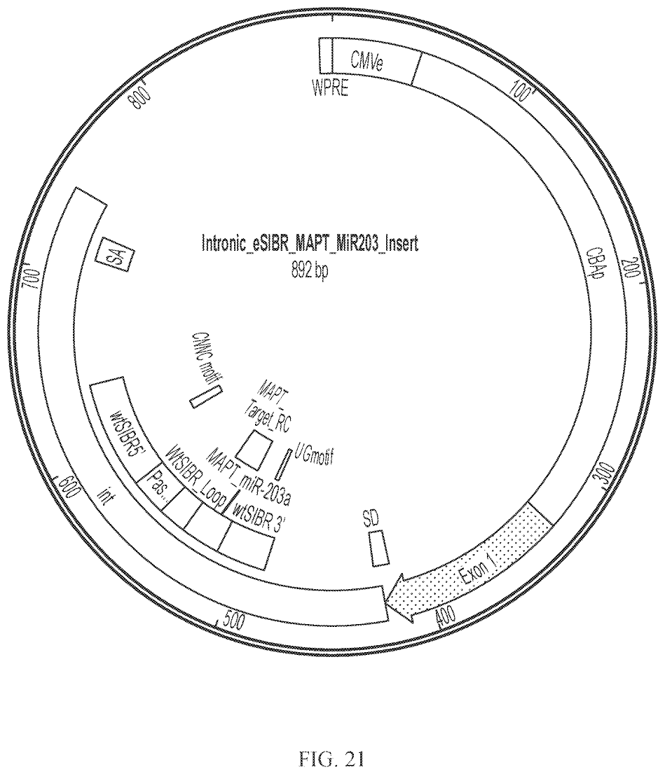

The effect of placement of ITR “D” sequence on cell transduction of rAAV vectors was investigated. HEK293 cells were transduced with Gcase-encoding rAAVs having 1) wild-type ITRs (e.g., “D” sequences proximal to the transgene insert and distal to the terminus of the ITR) or 2) ITRs with the “D” sequence located on the “outside” of the vector (e.g., “D” sequence located proximal to the terminus of the ITR and distal to the transgene insert), as shown in FIG. 20. Surprisingly, data indicate that rAAVs having the “D” sequence located in the “outside” position retain the ability to be packaged and transduce cells efficiently (FIG. 17).

### EQUIVALENTS

This Application incorporates by reference the contents of the following documents in their entirety: the International PCT Application referred to by Attorney Docket Number P1094.70002WO00, filed Oct. 3 2018; International PCT Application referred to by Attorney Docket Number P1094.70003WO00, filed Oct. 3 2018; Provisional Application Ser. Nos. 62/567,296, filed Oct. 3, 2017, entitled “GENE THERAPIES FOR LYSOSOMAL DISORDERS”; 62/567,311. filed Oct. 3, 2017. entitled “GENE THERAPIES FOR LYSOSOMAL DISORDERS”; 62/567,319. filed Oct. 3, 2017, entitled “GENE THERAPIES FOR LYSOSOMAL DISORDERS”; 62/567,301. filed Oct. 3, 2018, entitled “GENE THERAPIES FOR LYSOSOMAL DISORDERS”; 62/567,310, filed Oct. 3, 2017, entitled “GENE THERAPIES FOR LYSOSOMAL DISORDERS”; 62/567,303, filed Oct. 3, 2017, entitled “GENE THERAPIES FOR LYSOSOMAL DISORDERS”; and 62/567,305, filed Oct. 3, 2017, entitled “GENE THERAPIES FOR LYSOSOMAL DISORDERS”.

Having thus described several aspects of at least one embodiment of this invention, it is to be appreciated that various alterations, modifications, and improvements will readily occur to those skilled in the art, Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause. whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

### SEQUENCES

In some embodiments, an isolated nucleic acid, rAAV vector, or gene expression cassette encoding one or more gene products (e.g., a first, second and/or third gene product) comprises or consists of (or encodes a polypeptide having) a sequence set forth in any one of SEQ ID NO: 1-69. In some embodiments, a gene product is encoded by a portion (e.g., fragment) of any one of SEQ ID NOs: 1-69.

1-33. (canceled)

34. A method of treating a subject having or suspected of having Alzheimer disease (AD) or frontotemporal dementia (FTD), the method comprising administering to the subject a recombinant adeno-associated virus (rAAV) comprising:
(i) an adeno-associated virus (AAV) capsid protein; and
(ii) a rAAV vector comprising a nucleic acid having, in 5′ to 3′ order:
(a) a 5′ AAV inverted terminal repeat (ITR),
(b) a cytomegalovirus enhancer (CMV) enhancer,
(c) a chicken beta actin (CBA) promoter,
(d) a transgene insert encoding an inhibitory nucleic acid targeting microtubule-associated protein tau (MAPT), wherein the transgene insert comprises a nucleotide sequence as set forth in any one of SEQ ID NOS: 46-61,
(e) a Woodchuck Hepatitis Virus Posttranscriptional Regulatory Element (WPRE),
(f) a Bovine Growth Hormone polyA signal tail, and
(g) a 3′ AAV ITR.

35. The method of claim 34, wherein the administering comprises direct injection to the central nervous system (CNS) of the subject.

36. The method of claim 35, wherein the direct injection is intracerebral injection, intraparenchymal injection, intrathecal injection, intra-cisterna magna injection or a combination thereof.

37. The method of claim 35, wherein the direct injection to the CNS of the subject comprises convection enhanced delivery (CED).

38. The method of claim 34, wherein the administering comprises peripheral injection.

39. The method of claim 38, wherein the peripheral injection is intravenous injection.

40. The method of claim 34, wherein the AAV capsid protein is an AAV9 capsid protein.

41. A method for treating a subject having or suspected of having Parkinson's disease (PD) or Lewy Body Dementia (LBD), the method comprising administering to the subject a recombinant adeno-associated virus (rAAV) comprising:
(i) an adeno-associated virus (AAV) capsid protein; and
(ii) a rAAV vector comprising a nucleic acid having, in 5′ to 3′ order:
(a) a 5′ AAV inverted terminal repeat (ITR),
(b) a cytomegalovirus enhancer (CMV) enhancer,
(c) a chicken beta actin (CBA) promoter,
(d) a transgene encoding an inhibitory nucleic acid targeting α-Synuclein (SNCA), wherein the transgene comprises a nucleotide sequence as set forth in SEQ ID NO: 3;
(e) a Woodchuck Hepatitis Virus Posttranscriptional Regulatory Element (WPRE),
(f) a Bovine Growth Hormone polyA signal tail, and
(g) a 3′ AAV ITR.

42. The method of claim 41, wherein the administration comprises direct injection to the central nervous system (CNS) of the subject.

43. The method of claim 42, wherein the direct injection is intracerebral injection, intraparenchymal injection, intrathecal injection, intra-cisterna magna injection or a combination thereof.

44. The method of claim 41, wherein the direct injection to the CNS of the subject comprises convection enhanced delivery (CED).

45. The method of claim 41, wherein the administration comprises peripheral injection.

46. The method of claim 45, wherein the peripheral injection is intravenous injection.

47. The method of claim 41, wherein the AAV capsid protein is an AAV9 capsid protein.


SEQUENCE LISTING (69 sequences)

SEQ ID NO: 1 - DNA, 3022 bp, synthetic construct
  misc_feature 1..3022: note = Synthetic polynucleotide
  source 1..3022: mol_type = other DNA
attctggtgtgatccaggaacagctgtcttccagctctgaaagagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 2 - DNA, 6514 bp, synthetic construct
  misc_feature 1..6514: note = Synthetic polynucleotide
  source 1..6514: mol_type = other DNA
aggcgcggacgcaggttacagcagcgcttggcctctgctgatgccgtcgttatcctacccctcccccgtcccagctctacggcggccgcgcgctccaggccggtcgctccaccccccggctcccgggactgtggactccacgaccctgtcctcggccctgtccgcgccgaagcagcccgggactgcgcagcgccccgcgtgccgatcttttcctaattcagcagcgatttaaccaagagcctggaatattttaaggagtaataagagacatttacaaactattctctctgaagcctgctacctggaggcatcatctagataatcagaaccttggcttccacatcctcctcccttgtcttaactacaaacatttctttctgctgacttcaactcctcagacatgggaaagtctctttctcatttgcctttgcattcaagcaaagaagatgcttatgatggagtcacatctgaaaacatgaggaatggactggttaatagtgaagtccataatgaagatggaagaaatggagatgtctctcagtttccatatgtggaatttacaggaagagatagtgtcacctgccctacttgtcagggaacaggaagaattcctagggggcaagaaaaccaactggtggcattgattccatatagtgatcagagattaaggccaagaagaacaaagctgtatgtgatggcttctgtgtttgtctgtctactcctttctggattggctgtgtttttccttttccctcgctctatcgacgtgaaatacattggtgtaaaatcagcctatgtcagttatgatgttcagaagcgtacaatttatttaaatatcacaaacacactaaatataacaaacaataactattactctgtcgaagttgaaaacatcactgcccaagttcaattttcaaaaacagttattggaaaggcacgcttaaacaacataaccattattggtccacttgatatgaaacaaattgattacacagtacctaccgttatagcagaggaaatgagttatatgtatgatttctgtactctgatatccatcaaagtgcataacatagtactcatgatgcaagttactgtgacaacaacatactttggccactctgaacagatatcccaggagaggtatcagtatgtcgactgtggaagaaacacaacttatcagttggggcagtctgaatatttaaatgtacttcagccacaacagtaaaaactggaagagatggatttaaagaagaaatatctattgatatttcctatactctcaatgaagaggtatttcctaataggagaccttaaattgaacaaacctaaagtttacacttctaagagtacagttaaaagtatgtggacctgcagttcttgtaactctccactctgtgttaatgatatatttgtactaggatcttttacttgaatctaaatttactggttgatttccttctccagcctatcccctacagggaaaagctgatacttcccctatagtacaataaataattatttaaaagtcatagctccagtcactactgaaaacataattttggtgataaacataatttgagaaacttaatttctgaatgtttttatagaaaattactgaaagtctattactcatggaagacttttaaagaataaccttttttcctgttttataaattcccattgttatatggtagtatttcagctacacaatattttagcttttagctagacatttatagcttttcatttgttgaaatggtaatcatctgcatgtttttgtcacttatttcaggttagtgattgcctaacacttataagccaaaataatctttgcaaaattccatacctaaaattttgaaagcccctaatgttttcacacatctttctgtattagttatagttttgtgaaatctttgtgtgatcttcaaacattatcatttaatgtacaatactgtaaataaactgtgcatggcttttatacagctttagtaaatgtcaaataaagtggtacagactcattacaacaagtttctcataaaaatacaataaataggaaaatgaaattcagaaacccatagactgggaataggttccagttacagcttggatctggcataaaataaatttgaaataaaatattttgatgctccatttttttatgttgcttttcatactaaagaatggtgtagacatgttttgcaactgttaggtacccagttatcaattttatcaatgttttagaggaggaaattatttttttggtagaaattgttcaagaaatccttaattgaatgtcattaaatgatggtggccaaaataaaacctatttagaaatttaatcactttgcacatcacttggaatatgatgcctctagtagttacttttttatagttttctacttttggttttatttaaaattgttttcaaatatagattattgacttattcaactttgctgttttatattttcagtatcatttttcatttttttttttttttgtcttttcacttaccaagttctagggacatttaaaatatgtactaagtgtaggagtggttatgataccaaaaaatgtagctgggttgagattaatttcgttctgttttctcatgacagaaatcaggtttccctttccccacccctaagtgcctaacttaggtctgaaacagcctgtttattagtctgactctctcaaccataaaacataagctttatttaattctgcctttaaacacactcaggtttccccttaattttcatattattttctgcaagttttcttgagtatcttcaattcgttgaatgtggtttttggtttttttttgttttaacactagtcttcccttaattcattgctaactcaagccatccttactattaaacccaaatcagtcctttaagttcattatggcctttctagtatttaaaaaaaaaaaaatcattttcatttttcttctgctacgtttcctgactactactgcatacttctctgatacaggttctgtttgtattttttatatcattctcattttctcatttgacatgatctatgtctatatatgatataggtccccttttgtctcaaaatttttaattatgtgacttcaaaaatcacctgtatctgtagtagggcttccaaatctgcttctccatatgtgaccagtcacctgtctgctttcacatttagctagtgaactacacatttactaaaatgtgtaaattttacacatttagtgactgtgtaaaataaaaaaaaagttattttatcatatcctttctattatgttcccatcctgtcctcatgtcccatttactttattatcaccattcatttcttcaaaattatcttttagatacgctcatacaaaaatcaatccttgttttcttgcttgtgtcttttaaccttggaaaattacatcgtgtaaattaaacagatttttctgatgatctgtgcttcttatatactattagagtgcatgatagtatctcctgaaaaggatggaaagtagaagcatttgcttttagtcacttaattttgaatcttttttcttcatcttttgaattaattttttttattatatctacttttagtggagtttgagtcagaaaaaaacaagaatttgaaacaagtaaaaagatagaagagaaataaagatggtatgtgactactttcagagagagttaagtaactgtcagaataagcctggaacaaaacaggctgtaaattaataaaactacaaacacacattcaggtgaagcagaagtatagccataaaacatctagaaagagtgaatgaggcttttagcttttcttaggtcaatgtccagtgtgcttttttccatgggaataggataggtattaatacgcttttctaaactgctctcagaccttatccagaggacatggtaaagatatgttacagaaatttttctgatacttcctggaataactttaagttacaccctagtagactggtcattctaataaaatccagtactataacaaacctctgtatgttgatagcacattggccctttttagagttctttcctatgtttttcttacgtgatttcccacagttccatgagtccaacaaaggagagtgataggctccttatcttttagaagaggaaggaaaggcatgaagaagttgagggactggctgaagatcacgtacttactaagtagtacaactggagcaagatcaagtatctctgtctcccatatctgtgttctatcatttaaaatatatattggaaatccctgctgactcagattggtatgattaaaaatgagaggaaagttcaaatagttagtagtgacaaactaatactgctggactaagattttggtagcattgttttctaaaatattttaaatggagaatgaacacttataaaatgctttggaacataatctttagcttaattttctgttaaaatttagtaccccttcatcattccaataaagataagactgatccattgtctaaggaaattatttataaataatagagattaatttatttgagatttgaaataagaatagtatgaaaatattagataccacataaattgtttgaaattactgaataaccatcttaagtatggaacatttaaatggctatattttatttgtgtacagtttttctgtgccttgttaggccagtgaagcaattattttctctaagaaaatgacaataaaatataacacacttcagattgtctgatttacagtttggaaaggacaccgcaatgttcaaataggtaggagaccatcaaaaacacaattaaagtaacatattaggagacttgaaacttcagcctaataaatccttcatggttcttagccttattattgtgatataattctagatattttcttggagggcatgtgcccaactctcccgcaccccattttgtttgtcttttaaagttcttagaataaacagttctttatataataattatattttatttaagaaaatagtttgttaggtactttttaaaagatgtaaatttttaaatttacaaatacatatgggtctttgataagcaataggaattgaattacaagttactagggttataagcaaaaggttgcttaccataatgtcattaggtcacgatttttagctcacatctggaagcagcaactacttggctcaagtacatataagagtaattagttttattctctcttttttataaaatcgggtttcagatgagatgtttatcttagactattttagggaaaaattttacatgtttgagatggtggagtaaaaagactgttaaacatttcttttaaaaaattatttttacattacaacaatatatttatgatgtgttcagatcaaaaatttaacttctgtgtcccagatctactttcaaagtgagattttcacttgtcagcttaaatttctgactagaactaacatttgtgtatttttgtgcttagtcggaatacaaatttcacagtggatttttgaagtttgtccttaaattggataaaatcaagtgattaaagttactaaagagataaaaatggtaatttccatttttaaaagtaatttggttgtgtttatagttatttgtacaagtatttatcacagactctaaattgaaaaatgtagtatgatctatatttgaccctaaaaatgttggattaatttaacaaatatggcagatttttcataactaagtcttaagtcttctaaaaggaagctgttacccttctgtttttaattacattaattgaaatgtgttttaagagatacaatttcagcatattttatatattaaaaaacaaaaaaggattagtattgagccagtggccaaaaggtaatattactaccatgtagactgttatagttcaaattgtcccacttcacccagaattttagaaactagaagtctgggaggtactatatcagctgtagttgggtaattccaagtgctgatagtactattcatcttttttattattgtgtcagatgaaacaaatgccaagttgcaaaatatgcagatttttattatataatggttttaggcataaattattaacaagccatgccttatgtgtttcatcttatatttttctttagaactaaactataacagattttggaaaatgatttgacgtgcttgctcacttgattgacttggtcagatatttgaatgatggtattacctagattctaatccttgattctagttatataataaataatatagaatatgaaaatatgtttgggcatttactgtttatattatgtagtagcctccatcatgacacacttactacatttatgaattgagcagttctgtaattgtaattattattgctgttcatgtaacaaaacatgcttataatagcaaacaaatagaaatgcccccaaaatgctatttttttaattcagttataactgttactcttgtagttgtgtatgacgcaataaaatttgtaaaaaaatttcagcatgaaaaataaaatttgtatcacttatgta

SEQ ID NO: 3 - DNA, 23 bp, synthetic construct
  misc_feature 1..23: note = Synthetic polynucleotide
  source 1..23: mol_type = other DNA
tggaagacttcgagatacactgt

SEQ ID NO: 4 - DNA, 23 bp, synthetic construct
  misc_feature 1..23: note = Synthetic polynucleotide
  source 1..23: mol_type = other DNA
acagtgtatctcgaagtcttcca

SEQ ID NO: 5 - DNA, 21 bp, synthetic construct
  misc_feature 1..21: note = Synthetic polynucleotide
  source 1..21: mol_type = other DNA
tttagaaataagtggtagtca

SEQ ID NO: 6 - DNA, 21 bp, synthetic construct
  misc_feature 1..21: note = Synthetic polynucleotide
  source 1..21: mol_type = other DNA
tgactaccacttatttctaaa

SEQ ID NO: 7 - DNA, 19 bp, synthetic construct
  misc_feature 1..19: note = Synthetic polynucleotide
  source 1..19: mol_type = other DNA
agggtatcaagactacgaa

SEQ ID NO: 8 - DNA, 19 bp, synthetic construct
  misc_feature 1..19: note = Synthetic polynucleotide
  source 1..19: mol_type = other DNA
ttcgtagtcttgataccct

SEQ ID NO: 9 - DNA, 24 bp, synthetic construct
  misc_feature 1..24: note = Synthetic polynucleotide
  source 1..24: mol_type = other DNA
gtgtactaggatcttttacttgaa

SEQ ID NO: 10 - DNA, 24 bp, synthetic construct
  misc_feature 1..24: note = Synthetic polynucleotide
  source 1..24: mol_type = other DNA
ttcaagtaaaagatcctagtacac

SEQ ID NO: 11 - DNA, 267 bp, synthetic construct
  misc_feature 1..267: note = Synthetic polynucleotide
  source 1..267: mol_type = other DNA
gtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaa

SEQ ID NO: 12 - DNA, 267 bp, synthetic construct
  misc_feature 1..267: note = Synthetic polynucleotide
  source 1..267: mol_type = other DNA
tttgctacatgcaaagggcaggagactgatttgtgaactctggtgttggtgcctctggcagagaggggggtcctgagcccggatgccggcgtccccgttcaagtaaaagatcctagtacacattgggacctgcactgtactaggatcttttacttgaacgaggccacggccttcgccagggccttggtcctcctgctcttggctcctgtggcttcattccctcgggagagaatttctgaccccagccctgggaccttgtgatatcac

SEQ ID NO: 13 - DNA, 19 bp, synthetic construct
  misc_feature 1..19: note = Synthetic polynucleotide
  source 1..19: mol_type = other DNA
tattagatctgatggccgc

SEQ ID NO: 14 - DNA, 20 bp, synthetic construct
  misc_feature 1..20: note = Synthetic polynucleotide
  source 1..20: mol_type = other DNA
ctccatcactaggggttcct

SEQ ID NO: 15 - DNA, 60 bp, synthetic construct
  misc_feature 1..60: note = Synthetic polynucleotide
  source 1..60: mol_type = other DNA
agctctgggtatttaagcccgagtgagcacgcagggtctccattttgaagcgggaggtta

SEQ ID NO: 16 - DNA, 145 bp, synthetic construct
  misc_feature 1..145: note = AAV2 ITR
  source 1..145: mol_type = other DNA; note = AAV2 ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - DNA, 152 bp, synthetic construct
  misc_feature 1..152: note = Synthetic polynucleotide
  source 1..152: mol_type = other DNA
ctggaggcttgctttgggctgtatgctgtggaagacttcgagatacactgtttttggcctctgactgaacagtgttctgaagtcttccacaggacacaaggccctttatcagcactcacatggaacaaatggccaccgtgggaggatgacaa

SEQ ID NO: 18 - DNA, 152 bp, synthetic construct
  misc_feature 1..152: note = Synthetic polynucleotide
  source 1..152: mol_type = other DNA
ttgtcatcctcccacggtggccatttgttccatgtgagtgctgataaagggccttgtgtcctgtggaagacttcagaacactgttcagtcagaggccaaaaacagtgtatctcgaagtcttccacagcatacagcccaaagcaagcctccag

SEQ ID NO: 19 - DNA, 152 bp, synthetic construct
  misc_feature 1..152: note = Synthetic polynucleotide
  source 1..152: mol_type = other DNA
ttgtcatcctcccacggtggccatttgttccatgtgagtgctagtaacaggccttgtgtccttttagaaataagtggtagtcacatctgtggcttcacttgactaccacttatttctaaagacaacagcatacagccttcagcaagcctcca

SEQ ID NO: 20 - DNA, 152 bp, synthetic construct
  misc_feature 1..152: note = Synthetic polynucleotide
  source 1..152: mol_type = other DNA
tggaggcttgctgaaggctgtatgctgttgtctttagaaataagtggtagtcaagtgaagccacagatgtgactaccacttatttctaaaaggacacaaggcctgttactagcactcacatggaacaaatggccaccgtgggaggatgacaa

SEQ ID NO: 21 - DNA, 179 bp, synthetic construct
  misc_feature 1..179: note = Synthetic polynucleotide
  source 1..179: mol_type = other DNA
tggaggcttgctgaaggctgtatgctgttgtcctcgagtgagcgtagggtatcaagactacgaatactgtaaagccacagatgggtgttcgtagtcttgatacccttcgcctactagaggacacaaggcctgttactagcactcacatggaacaaatggccaccgtgggaggatgacaa

SEQ ID NO: 22 - DNA, 179 bp, synthetic construct
  misc_feature 1..179: note = Synthetic polynucleotide
  source 1..179: mol_type = other DNA
ttgtcatcctcccacggtggccatttgttccatgtgagtgctagtaacaggccttgtgtcctctagtaggcgaagggtatcaagactacgaacacccatctgtggctttacagtattcgtagtcttgataccctacgctcactcgaggacaacagcatacagccttcagcaagcctcca

SEQ ID NO: 23 - DNA, 10960 bp, synthetic construct
  misc_feature 1..10960: note = Synthetic polynucleotide
  source 1..10960: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtcctggtggcgaggggaggggggtggtcctcgaacgccttgcagaactggcctggatacagagtggaccggctggccccatctggaagacttcgagatacactgttgtcttactgcgctcaacagtgtatctcgaagtcttccaaatggtgccagccatcgcagcggggtgcaggaaatgggggcagcccccctttttggctatccttccacgtgttcttttttgtatcttttgtgtttcctagaaaacatctcagtcaccacctttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 24 - DNA, 10013 bp, synthetic construct
  misc_feature 1..10013: note = Synthetic polynucleotide
  source 1..10013: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggacgtgttcatgaagggcctgagcaaggccaaggagggcgtggtggccgccgccgagaagaccaagcagggcgtggccgaggccgccggcaagaccaaggagggcgtgctgtacgtgggcagcaagaccaaggagggcgtggtgcacggcgtggccaccgtggccgagaagaccaaggagcaggtgaccaacgtgggcggcgccgtggtgaccggcgtgaccgccgtggcccagaagaccgtggagggcgccggcagcatcgccgccgccaccggcttcgtgaagaaggaccagctgggcaagaacgaggagggcgccccccaggagggcatcctggaggacatgcccgtggaccccgacaacgaggcctacgagatgcccagcgaggagggctaccaggactacgagcccgaggcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgctgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 25 - DNA, 10849 bp, synthetic construct
  misc_feature 1..10849: note = Synthetic polynucleotide
  source 1..10849: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgtggaagacttcgagatacactgtttttggcctctgactgaacagtgttctgaagtcttccacaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 26 - DNA, 11231 bp, synthetic construct
  misc_feature 1..11231: note = Synthetic polynucleotide
  source 1..11231: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcttgtcatcctcccacggtggccatttgttccatgtgagtgctagtaacaggccttgtgtccttttagaaataagtggtagtcacatctgtggcttcacttgactaccacttatttctaaagacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgacattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 27 - DNA, 10876 bp, synthetic construct
  misc_feature 1..10876: note = Synthetic polynucleotide
  source 1..10876: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcctcgagtgagcgtagggtatcaagactacgaatactgtaaagccacagatgggtgttcgtagtcttgatacccttcgcctactagaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 28 - DNA, 10849 bp, synthetic construct
  misc_feature 1..10849: note = Synthetic polynucleotide
  source 1..10849: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtctttagaaataagtggtagtcaagtgaagccacagatgtgactaccacttatttctaaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 29 - DNA, 11188 bp, synthetic construct
  misc_feature 1..11188: note = Synthetic polynucleotide
  source 1..11188: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactattagatctgatggccgcgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 30 - DNA, 11174 bp, synthetic construct
  misc_feature 1..11174: note = Synthetic polynucleotide
  source 1..11174: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtcggtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 31 - DNA, 10841 bp, synthetic construct
  misc_feature 1..10841: note = Synthetic polynucleotide
  source 1..10841: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgagggtatcaagactacgaattttggcctctgactgattcgtagcttataccctcaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 32 - DNA, 11187 bp, synthetic construct
  misc_feature 1..11187: note = Synthetic polynucleotide
  source 1..11187: mol_type = other DNA
ctagaaccggtgacgtctcccatggtgaagcttggatctgaattcggtacctagttataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtcttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcgtggtgactgagatgttttctaggaaacacaaaagatacaaaaaagaacacgtggaaggatagccaaaaaggggggctgcccccatttcctgcaccccgctgcgatggctggcaccatttggaagacttcgagatacactgttgagcgcagtaagacaacagtgtatctcgaagtcttccagatggggccagccggtccactctgtatccaggccagttctgcaaggcgttcgaggaccacccccctcccctcgccaccagggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaatccctaggtt

SEQ ID NO: 33 - DNA, 10996 bp, synthetic construct
  misc_feature 1..10996: note = Synthetic polynucleotide
  source 1..10996: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtcctagtaggcgaagggtatcaagactacgaacacccatctgtggctttacagtattcgtagtcttgataccctacgctcactcgaggtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 34 - DNA, 10845 bp, synthetic construct
  misc_feature 1..10845: note = Synthetic polynucleotide
  source 1..10845: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtctggaggcttgctttgggctgtatgctgtttagaaataagtggtagtcattttggcctctgactgatgactacactatttctaaacaggacacaaggccctttatcagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacagtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 35 - DNA, 11320 bp, synthetic construct
  misc_feature 1..11320: note = Synthetic polynucleotide
  source 1..11320: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgtgatatcacaaggtcccagggctggggtcagaaattctctcccgagggaatgaagccacaggagccaagagcaggaggaccaaggccctggcgaaggccgtggcctcgttcaagtaaaagatcctagtacagtgcaggtcccaatgtgtactaggatcttttacttgaacggggacgccggcatccgggctcaggacccccctctctgccagaggcaccaacaccagagttcacaaatcagtctcctgccctttgcatgtagcaaagcagccctaggaatgcatctagacaattgtactaaccttcttctctttcctctcctgacagtccggaaagccaccatggaattcagcagccccagcagagaggaatgccccaagcctctgagccgggtgtcaatcatggccggatctctgacaggactgctgctgcttcaggccgtgtcttgggcttctggcgctagaccttgcatccccaagagcttcggctacagcagcgtcgtgtgcgtgtgcaatgccacctactgcgacagcttcgaccctcctacctttcctgctctgggcaccttcagcagatacgagagcaccagatccggcagacggatggaactgagcatgggacccatccaggccaatcacacaggcactggcctgctgctgacactgcagcctgagcagaaattccagaaagtgaaaggcttcggcggagccatgacagatgccgccgctctgaatatcctggctctgtctccaccagctcagaacctgctgctcaagagctacttcagcgaggaaggcatcggctacaacatcatcagagtgcccatggccagctgcgacttcagcatcaggacctacacctacgccgacacacccgacgatttccagctgcacaacttcagcctgcctgaagaggacaccaagctgaagatccctctgatccacagagccctgcagctggcacaaagacccgtgtcactgctggcctctccatggacatctcccacctggctgaaaacaaatggcgccgtgaatggcaagggcagcctgaaaggccaacctggcgacatctaccaccagacctgggccagatacttcgtgaagttcctggacgcctatgccgagcacaagctgcagttttgggccgtgacagccgagaacgaaccttctgctggactgctgagcggctacccctttcagtgcctgggctttacacccgagcaccagcgggactttatcgcccgtgatctgggacccacactggccaatagcacccaccataatgtgcggctgctgatgctggacgaccagagactgcttctgccccactgggctaaagtggtgctgacagatcctgaggccgccaaatacgtgcacggaatcgccgtgcactggtatctggactttctggcccctgccaaggccacactgggagagacacacagactgttccccaacaccatgctgttcgccagcgaagcctgtgtgggcagcaagttttgggaacagagcgtgcggctcggcagctgggatagaggcatgcagtacagccacagcatcatcaccaacctgctgtaccacgtcgtcggctggaccgactggaatctggccctgaatcctgaaggcggccctaactgggtccgaaacttcgtggacagccccatcatcgtggacatcaccaaggacaccttctacaagcagcccatgttctaccacctgggacacttcagcaagttcatccccgagggctctcagcgcgttggactggtggcttcccagaagaacgatctggacgccgtggctctgatgcaccctgatggatctgctgtggtggtggtcctgaaccgcagcagcaaagatgtgcccctgaccatcaaggatcccgccgtgggattcctggaaacaatcagccctggctactccatccacacctacctgtggcgtagacaggagggcagaggaagtcttctgacatgcggagacgtggaagagaatcccggccctatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcaataaaatatctttattttcattacatctgtgtgttggttttttgtgtggagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcgtacggtctcgaggaattcctgcaggataacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgcagaccagcctggccaacatgatgaaaccctctctactaataataaaatcagtagaactactcaggactactttgagtgggaagtccttttctatgaagacttctttggccaaaattaggctctaaatgcaaggagatagtgcatcatgcctggctgcacttactgataaatgatgttatcaccatctttaaccaaatgcacaggaacaagttatggtactgatgtgctggattgagaaggagctctacttccttgacaggacacatttgtatcaacttaaaaaagcagatttttgccagcagaactattcattcagaggtaggaaacttagaatagatgatgtcactgattagcatggcttccccatctccacagctgcttcccacccaggttgcccacagttgagtttgtccagtgctcagggctgcccactctcagtaagaagccccacaccagcccctctccaaatatgttggctgttccttccattaaagtgaccccactttagagcagcaagtggatttctgtttcttacagttcaggaaggaggagtcagctgtgagaacctggagcctgagatgcttctaagtcccactgctactggggtcagggaagccagactccagcatcagcagtcaggagcactaagcccttgccaacatcctgtttctcagagaaactgcttccattataatggttgtccttttttaagctatcaagccaaacaaccagtgtctaccattattctcatcacctgaagccaagggttctagcaaaagtcaagctgtcttgtaatggttgatgtgcctccagcttctgtcttcagtcactccactcttagcctgctctgaatcaactctgaccacagttccctggagcccctgccacctgctgcccctgccaccttctccatctgcagtgctgtgcagccttctgcactcttgcagagctaataggtggagacttgaaggaagaggaggaaagtttctcataatagccttgctgcaagctcaaatgggaggtgggcactgtgcccaggagccttggagcaaaggctgtgcccaacctctgactgcatccaggtttggtcttgacagagataagaagccctggcttttggagccaaaatctaggtcagacttaggcaggattctcaaagtttatcagcagaacatgaggcagaagaccctttctgctccagcttcttcaggctcaaccttcatcagaatagatagaaagagaggctgtgagggttcttaaaacagaagcaaatctgactcagagaataaacaacctcctagtaaactacagcttagacagagcatctggtggtgagtgtgctcagtgtcctactcaactgtctggtatcagccctcatgaggacttctcttctttccctcatagacctccatctctgttttccttagcctgcagaaatctggatggctattcacagaatgcctgtgctttcagagttgcattttttctctggtattctggttcaagcatttgaaggtaggaaaggttctccaagtgcaagaaagccagccctgagcctcaactgcctggctagtgtggtcagtaggatgcaaaggctgttgaatgccacaaggccaaactttaacctgtgtaccacaagcctagcagcagaggcagctctgctcactggaactctctgtcttctttctcctgagccttttcttttcctgagttttctagctctcctcaaccttacctctgccctacccaggacaaacccaagagccactgtttctgtgatgtcctctccagccctaattaggcatcatgacttcagcctgaccttccatgctcagaagcagtgctaatccacttcagatgagctgctctatgcaacacaggcagagcctacaaacctttgcaccagagccctccacatatcagtgtttgttcatactcacttcaacagcaaatgtgactgctgagattaagattttacacaagatggtctgtaatttcacagttagttttatcccattaggtatgaaagaattagcataattccccttaaacatgaatgaatcttagattttttaataaatagttttggaagtaaagacagagacatcaggagcacaaggaatagcctgagaggacaaacagaacaagaaagagtctggaaatacacaggatgttcttggcctcctcaaagcaagtgcaagcagatagtaccagcagccccaggctatcagagcccagtgaagagaagtaccatgaaagccacagctctaaccaccctgttccagagtgacagacagtccccaagacaagccagcctgagccagagagagaactgcaagagaaagtttctaatttaggttctgttagattcagacaagtgcaggtcatcctctctccacagctactcacctctccagcctaacaaagcctgcagtccacactccaaccctggtgtctcacctcctagcctctcccaacatcctgctctctgaccatcttctgcatctctcatctcaccatctcccactgtctacagcctactcttgcaactaccatctcattttctgacatcctgtctacatcttctgccatactctgccatctaccataccacctcttaccatctaccacaccatcttttatctccatccctctcagaagcctccaagctgaatcctgctttatgtgttcatctcagcccctgcatggaaagctgaccccagaggcagaactattcccagagagcttggccaagaaaaacaaaactaccagcctggccaggctcaggagtagtaagctgcagtgtctgttgtgttctagcttcaacagctgcaggagttccactctcaaatgctccacatttctcacatcctcctgattctggtcactacccatcttcaaagaacagaatatctcacatcagcatactgtgaaggactagtcatgggtgcagctgctcagagctgcaaagtcattctggatggtggagagcttacaaacatttcatgatgctccccccgctctgatggctggagcccaatccctacacagactcctgctgtatgtgttttcctttcactctgagccacagccagagggcaggcattcagtctcctcttcaggctggggctggggcactgagaactcacccaacaccttgctctcactccttctgcaaaacaagaaagagctttgtgctgcagtagccatgaagaatgaaaggaaggctttaactaaaaaatgtcagagattattttcaaccccttactgtggatcaccagcaaggaggaaacacaacacagagacattttttcccctcaaattatcaaaagaatcactgcatttgttaaagagagcaactgaatcaggaagcagagttttgaacatatcagaagttaggaatctgcatcagagacaaatgcagtcatggttgtttgctgcataccagccctaatcattagaagcctcatggacttcaaacatcattccctctgacaagatgctctagcctaactccatgagataaaataaatctgcctttcagagccaaagaagagtccaccagcttcttctcagtgtgaacaagagctccagtcaggttagtcagtccagtgcagtagaggagaccagtctgcatcctctaattttcaaaggcaagaagatttgtttaccctggacaccaggcacaagtgaggtcacagagctcttagatatgcagtcctcatgagtgaggagactaaagcgcatgccatcaagacttcagtgtagagaaaacctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgagggcgcgccaagtcgacgtccggcagtc

SEQ ID NO: 36 - DNA, 3793 bp, synthetic construct
  misc_feature 1..3793: note = Synthetic polynucleotide
  source 1..3793: mol_type = other DNA
tctcattagaagtgaggcggggccggccaaatcgaatggacaccgggtaattagcagggttacccagatactccagcacctctttcccgtcggccgtgtacctgccattcacgtccatgccattgatggccagcactgcatgacccactgcagaggtgaagctaacggtcagcgaaggtgcagcccggggattccgccgaggggacaagggacccgacacaaccccttttcccccaaccccgcacctacaaccagcccacttctacagcactggggccctcccacccccgcacccgccacgggcccgagcctagcccacctcggatgccgtcccgctggccgaaagcaaccaacacacgctcatcgtgtagcttgagcagcagatccagcggataactgaaagttttctcagcctcagcccgtggcgcgtagctgtccaactggtaaatcaagccgccagctttgttcaccacatacacactaaaaatcgccatcgctgccttgccgctcggaaactggtattcagcctctacccgacggcccctccccggaaccgcatcacagcacttgccgccggccccaccccagcctcctcctcctcctcctcctcctcccgcgccccccgtgcagccacctgctgcacttgcgcactgggagcgacacgctcgggcataagtagtgccgaaaagttagctgccgagacctggtggattgcttttcgtttatcagtgcaggaaaacagcgctatagtactgcgtcacaactagcgcagactccggcagtatttaggcggtgcggcttgggaactagaatccacttcctgtcttccgcctcaggctagagggcgagcgcttcgccgtgggacttcttctgcctggctccgcctcttgccccggaagtactcacagcggacggtggtttttgggcccgtttctgagcagcgcttcctttttgtccgacatcttgacgaggctgcggtgtctgctgctattctccgagcttcgcaatggtaagcttcaggggtgtgaagtcgccggcgttcttgggtttgaggactcagtggggagagccttcggcgggagcgctccttggcctgccggcctcggttgcagggcgggcgcggttattgcttggcccatgtgctctggtggtggagtttgcgggggctgagggcgcagtattaggggactttggcgctatttgaggacctggttgcattcccgctgccctcctacagccgcctaaggacgacaagaagaagaaggacgctggaaagtcggccaagaaagacaaagacccagtgaacaaatccgggggcaaggccaaaaagaaggtagaaataagacctctctgaaagagactaggggtaactctctcgtaatcctctagtaataggtaacttgtatagtaagtggtttttcaggtgtagatttctagagtcaaaatgtgagagtttatcttcccgtcaccactcgttctttttcccattaggatcatgaaaatgggtctgttgtgcgaagtgtctgccgctgtgcctgctgtgttatttttaactgatctagtggggctcggcccctgtttgaaggccaaaaacgtgtcggtgttttttttttgtttttgttttagtaatgtgtaatttatccttgataacggtggaacagatttctctgacgcagattactcgagagggaaagggtgcttctgccagaaatactaacttgtttctgttttgttttggtgagcagaagtggtccaaaggcaaagttcgggacaagctcaataacttagtcttgtttgacaaagctacctatgataaactctgtaaggaagttcccaactataaacttataaccccagctgtggtctctgagagactgaagattcgaggctccctggccagggcagcccttcaggagctccttagtaaaggtgaggggtgtatcctacatgtgtgtttttgtaggttaaattgtcttgaccatgttaagcatcttcagtggttttgctggaaaagcagaattaaaaaaaaaaagcgtggcttgaccattggctgttagtaatgtaattctgacgtcttactcctgatcctgagatgaattctcagggttcttagccacttttgtgccgtggaccctgtggcagtttagtgaagcccaaggatcttttatgtttcgagtaaatggatgcatagaattacagggacaaccgtttttgaaataattagattactattttgaaacaactttgaaaatgtttaaaacctttatggtaaatattttgttgatgtattaaattttaaaaccagaaatttagtacggtctactcagtagtatggtctgattaccataattccacaataataaggctcagctaactatagtgactgaacgtctataattctagcactttgggaggccaaggcgggtgaatcaacggaggtcaggagttaaagaccagcctggccaatatggtgaaaacctgctctactgaaagttagctggacgtgggggcacacgtctgtaatcccagctactcaggatgctgaggcatgaggatcccttgaacccaggagatggaggtggcagtgagccgagatgacaccactgcactccagccttagtgacagcaaaagactgtctcagaaagggggggggggtggaagataatggagccctaatttaaaggaaaagtaaggatagatgatccgttaaaaacttggattctcggttaccgaacgtcagattaagcaattctggagccaggtgcagtggtacccttgtatttctagctacttgggaggccaaagcaggaggatcatttgagccaaggagttttaagaccattctgggcacctctgagagaactctgtctttttgttttccttttctttaaatagagatgcggttttgccatgttgcccaggctggtctcctgggctcaagagatccacctgtccaaagtgctgggattacaggcatgagcctctgcacccggccaaaacaaaccttactagagtctcattctgttgcccaggttggagtgcggaggggcagtcttggctcaatgcaaccaccaattcctgggttcaggtggtcctcacctcagcttcccaagtagctggaattacaagcatgtgccaccatgcccagctaatttttgtatttttggtagagatggggtttcaccttgttggccaggctggtgtgcaactccttacctcaagctatctgcccgtctccacctcccaaagcagtgggattataagcatgagccaccgcgcccagccaaaaaccttactagtttctattgtagcatctgttaagcatctcatcgtgctattctctccccctaggacttatcaaactggtttcaaagcacagagctcaagtaatttacaccagaaataccaagggtggagatgctccagctgctggtgaagatgcatgaataggtgagtaggaatgtgtgggctcatggtgtaggaggtagatacaaagctttatggttctgattcttttaatttttttttacaggtccaaccagctgtacatttggaaaaataaaactttattaaatcaaatgaatgagtatgtctgtttcctaagaaagacaatgataaagaatttggtggaaggtataataggggtttgttgactttgcttttagcctcatggtagttggtagagagcatgattagcttttttctgtatgtgactgcttcttcattgctgcagcttcagttttgaattgatgtctgaaaggaaataaagggttaacacgatgatgaagggtg

SEQ ID NO: 37 - DNA, 6762 bp, synthetic construct
  misc_feature 1..6762: note = Synthetic polynucleotide
  source 1..6762: mol_type = other DNA
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 38 - DNA, 22 bp, synthetic construct
  misc_feature 1..22: note = Synthetic polynucleotide
  source 1..22: mol_type = other DNA
ataccttccaccaaattcttta

SEQ ID NO: 39 - DNA, 22 bp, synthetic construct
  misc_feature 1..22: note = Synthetic polynucleotide
  source 1..22: mol_type = other DNA
taaagaatttggtggaaggtat

SEQ ID NO: 40 - DNA, 150 bp, synthetic construct
  misc_feature 1..150: note = Synthetic polynucleotide
  source 1..150: mol_type = other DNA
ctggaggcttgctttgggctgtatgctgataccttccaccaaattctttattttggcctctgactgataaagaattgtggaaggtatcaggacacaaggccctttatcagcactcacatggaacaaatggccaccgtgggaggatgacaa

SEQ ID NO: 41 - DNA, 150 bp, synthetic construct
  misc_feature 1..150: note = Synthetic polynucleotide
  source 1..150: mol_type = other DNA
ttgtcatcctcccacggtggccatttgttccatgtgagtgctgataaagggccttgtgtcctgataccttccacaattctttatcagtcagaggccaaaataaagaatttggtggaaggtatcagcatacagcccaaagcaagcctccag

SEQ ID NO: 42 - DNA, 21 bp, synthetic construct
  misc_feature 1..21: note = Synthetic polynucleotide
  source 1..21: mol_type = other DNA
ataagtcctttactaaggagc

SEQ ID NO: 43 - DNA, 21 bp, synthetic construct
  misc_feature 1..21: note = Synthetic polynucleotide
  source 1..21: mol_type = other DNA
gctccttagtaaaggacttat

SEQ ID NO: 44 - DNA, 148 bp, synthetic construct
  misc_feature 1..148: note = Synthetic polynucleotide
  source 1..148: mol_type = other DNA
ctggaggcttgctttgggctgtatgctgataagtcctttactaaggagcttttggcctctgactgagctccttgtaaggacttatcaggacacaaggccctttatcagcactcacatggaacaaatggccaccgtgggaggatgacaa

SEQ ID NO: 45 - DNA, 148 bp, synthetic construct
  misc_feature 1..148: note = Synthetic polynucleotide
  source 1..148: mol_type = other DNA
ttgtcatcctcccacggtggccatttgttccatgtgagtgctgataaagggccttgtgtcctgataagtccttacaaggagctcagtcagaggccaaaagctccttagtaaaggacttatcagcatacagcccaaagcaagcctccag

SEQ ID NO: 46 - DNA, 21 bp, synthetic construct
  misc_feature 1..21: note = Synthetic polynucleotide
  source 1..21: mol_type = other DNA
gattttgaagtcccgagccaa

SEQ ID NO: 47 - DNA, 21 bp, synthetic construct
  misc_feature 1..21: note = Synthetic polynucleotide
  source 1..21: mol_type = other DNA
ttggctcgggacttcaaaatc

SEQ ID NO: 48 - DNA, 148 bp, synthetic construct
  misc_feature 1..148: note = Synthetic polynucleotide
  source 1..148: mol_type = other DNA
ctggaggcttgctttgggctgtatgctggattttgaagtcccgagccaattttggcctctgactgattggctcggattcaaaatccaggacacaaggccctttatcagcactcacatggaacaaatggccaccgtgggaggatgacaa

SEQ ID NO: 49 - DNA, 148 bp, synthetic construct
  misc_feature 1..148: note = Synthetic polynucleotide
  source 1..148: mol_type = other DNA
ttgtcatcctcccacggtggccatttgttccatgtgagtgctgataaagggccttgtgtcctggattttgaatccgagccaatcagtcagaggccaaaattggctcgggacttcaaaatccagcatacagcccaaagcaagcctccag

SEQ ID NO: 50 - DNA, 23 bp, synthetic construct
  misc_feature 1..23: note = Synthetic polynucleotide
  source 1..23: mol_type = other DNA
ggaaatgttggatgtggccatgt

SEQ ID NO: 51 - DNA, 23 bp, synthetic construct
  misc_feature 1..23: note = Synthetic polynucleotide
  source 1..23: mol_type = other DNA
acatggccacatccaacatttcc

SEQ ID NO: 52 - DNA, 152 bp, synthetic construct
  misc_feature 1..152: note = Synthetic polynucleotide
  source 1..152: mol_type = other DNA
ctggaggcttgctttgggctgtatgctgggaaatgttggatgtggccatgtttttggcctctgactgaacatggcacaccaacatttcccaggacacaaggccctttatcagcactcacatggaacaaatggccaccgtgggaggatgacaa

SEQ ID NO: 53 - DNA, 152 bp, synthetic construct
  misc_feature 1..152: note = Synthetic polynucleotide
  source 1..152: mol_type = other DNA
ttgtcatcctcccacggtggccatttgttccatgtgagtgctgataaagggccttgtgtcctgggaaatgttggtgtgccatgttcagtcagaggccaaaaacatggccacatccaacatttcccagcatacagcccaaagcaagcctccag

SEQ ID NO: 54 - DNA, 45 bp, synthetic construct
  misc_feature 1..45: note = Synthetic polynucleotide
  source 1..45: mol_type = other DNA
ggtgcagataattaataagttcgcttattaattatctgcaccttc

SEQ ID NO: 55 - DNA, 45 bp, synthetic construct
  misc_feature 1..45: note = Synthetic polynucleotide
  source 1..45: mol_type = other DNA
gaaggtgcagataattaataagcgaacttattaattatctgcacc

SEQ ID NO: 56 - DNA, 139 bp, synthetic construct
  misc_feature 1..139: note = Synthetic polynucleotide
  source 1..139: mol_type = other DNA
tggaggcttgctgaaggctgtatgctgttgtcggtgcagataattaataagttcgcttattaattatctgcaccttcaggacacaaggcctgttactagcactcacatggaacaaatggccaccgtgggaggatgacaa

SEQ ID NO: 57 - DNA, 139 bp, synthetic construct
  misc_feature 1..139: note = Synthetic polynucleotide
  source 1..139: mol_type = other DNA
ttgtcatcctcccacggtggccatttgttccatgtgagtgctagtaacaggccttgtgtcctgaaggtgcagataattaataagcgaacttattaattatctgcaccgacaacagcatacagccttcagcaagcctcca

SEQ ID NO: 58 - DNA, 21 bp, synthetic construct
  misc_feature 1..21: note = Synthetic polynucleotide
  source 1..21: mol_type = other DNA
ttgtagactatttgcacactg

SEQ ID NO: 59 - DNA, 21 bp, synthetic construct
  misc_feature 1..21: note = Synthetic polynucleotide
  source 1..21: mol_type = other DNA
cagtgtgcaaatagtctacaa

SEQ ID NO: 60 - DNA, 152 bp, synthetic construct
  misc_feature 1..152: note = Synthetic polynucleotide
  source 1..152: mol_type = other DNA
ttgtcatcctcccacggtggccatttgttccatgtgagtgctagtaacaggccttgtgtcctttgtagactatttgcacactgcatctgtggcttcactcagtgtgcaaatagtctacaagacaacagcatacagccttcagcaagcctcca

SEQ ID NO: 61 - DNA, 152 bp, synthetic construct
  misc_feature 1..152: note = Synthetic polynucleotide
  source 1..152: mol_type = other DNA
tggaggcttgctgaaggctgtatgctgttgtcttgtagactatttgcacactgagtgaagccacagatgcagtgtgcaaatagtctacaaaggacacaaggcctgttactagcactcacatggaacaaatggccaccgtgggaggatgacaa

SEQ ID NO: 62 - DNA, 4321 bp, synthetic construct
  misc_feature 1..4321: note = Synthetic polynucleotide
  source 1..4321: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcggtgcagataattaataagttcgcttattaattatctgcaccttcaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 63 - DNA, 4552 bp, synthetic construct
  misc_feature 1..4552: note = Synthetic polynucleotide
  source 1..4552: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcaaaaaaattgtcatcctcccacggtggccatttgttccatgtgagtgctagtaacaggccttgtgtcctttgtagactatttgcacactgcatctgtggcttcactcagtgtgcaaatagtctacaagacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgacattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgtggaccctggtgagctgggtggccctgaccgccggcctggtggccggcacccgctgccccgacggccagttctgccccgtggcctgctgcctggaccccggcggcgccagctacagctgctgccgccccctgctggacaagtggcccaccaccctgagccgccacctgggcggcccctgccaggtggacgcccactgcagcgccggccacagctgcatcttcaccgtgagcggcaccagcagctgctgccccttccccgaggccgtggcctgcggcgacggccaccactgctgcccccgcggcttccactgcagcgccgacggccgcagctgcttccagcgcagcggcaacaacagcgtgggcgccatccagtgccccgacagccagttcgagtgccccgacttcagcacctgctgcgtgatggtggacggcagctggggctgctgccccatgccccaggccagctgctgcgaggaccgcgtgcactgctgcccccacggcgccttctgcgacctggtgcacacccgctgcatcacccccaccggcacccaccccctggccaagaagctgcccgcccagcgcaccaaccgcgccgtggccctgagcagcagcgtgatgtgccccgacgcccgcagccgctgccccgacggcagcacctgctgcgagctgcccagcggcaagtacggctgctgccccatgcccaacgccacctgctgcagcgaccacctgcactgctgcccccaggacaccgtgtgcgacctgatccagagcaagtgcctgagcaaggagaacgccaccaccgacctgctgaccaagctgcccgcccacaccgtgggcgacgtgaagtgcgacatggaggtgagctgccccgacggctacacctgctgccgcctgcagagcggcgcctggggctgctgccccttcacccaggccgtgtgctgcgaggaccacatccactgctgccccgccggcttcacctgcgacacccagaagggcacctgcgagcagggcccccaccaggtgccctggatggagaaggcccccgcccacctgagcctgcccgacccccaggccctgaagcgcgacgtgccctgcgacaacgtgagcagctgccccagcagcgacacctgctgccagctgaccagcggcgagtggggctgctgccccatccccgaggccgtgtgctgcagcgaccaccagcactgctgcccccagggctacacctgcgtggccgagggccagtgccagcgcggcagcgagatcgtggccggcctggagaagatgcccgcccgccgcgccagcctgagccacccccgcgacatcggctgcgaccagcacaccagctgccccgtgggccagacctgctgccccagcctgggcggcagctgggcctgctgccagctgccccacgccgtgtgctgcgaggaccgccagcactgctgccccgccggctacacctgcaacgtgaaggcccgcagctgcgagaaggaggtggtgagcgcccagcccgccaccttcctggcccgcagcccccacgtgggcgtgaaggacgtggagtgcggcgagggccacttctgccacgacaaccagacctgctgccgcgacaaccgccagggctgggcctgctgcccctaccgccagggcgtgtgctgcgccgaccgccgccactgctgccccgccggcttccgctgcgccgcccgcggcaccaagtgcctgcgccgcgaggccccccgctgggacgcccccctgcgcgaccccgccctgcgccagctgctgtgacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 64 - DNA, 23 bp, synthetic construct
  misc_feature 1..23: note = Synthetic polynucleotide
  source 1..23: mol_type = other DNA
aagagggtgttctctatgtaggc

SEQ ID NO: 65 - DNA, 22 bp, synthetic construct
  misc_feature 1..22: note = Synthetic polynucleotide
  source 1..22: mol_type = other DNA
gctcctccaacatttgtcactt

SEQ ID NO: 66 - DNA, 23 bp, synthetic construct
  misc_feature 1..23: note = Synthetic polynucleotide
  source 1..23: mol_type = other DNA
acacagtacctaccgttatagca

SEQ ID NO: 67 - DNA, 23 bp, synthetic construct
  misc_feature 1..23: note = Synthetic polynucleotide
  source 1..23: mol_type = other DNA
tgttgtcacagtaacttgcatca

SEQ ID NO: 68 - DNA, 19 bp, synthetic construct
  misc_feature 1..19: note = Synthetic polynucleotide
  source 1..19: mol_type = other DNA
ctgggctacactgagcacc

SEQ ID NO: 69 - DNA, 21 bp, synthetic construct
  misc_feature 1..21: note = Synthetic polynucleotide
  source 1..21: mol_type = other DNA
aagtggtcgttgagggcaatg